SEQUENCE LISTING (2 sequences)

SEQ ID NO: 1 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 2 - protein, 152 aa, Homo sapiens
MRGPGHPLLLGLLLVLGAAGRGRGGAEPREPADGQALLRLVVELVQELRKHHSAEHKGLQLLGRDCALGRAEAAGLGPSPEQRVEIVPRDLRMKDKFLKHLTGPLYFSPKCSKHFHRLYHNTRDCTIPAYYKRCARLLTRLAVSPVCMEDKQ

(19) United States
(12) Patent Application Publication
(10) Pub. No.: US 2025/0228918 A1
(43) Pub. Date: Jul. 17, 2025

(54) METHODS OF CONTROLLING BODY WEIGHT AND/OR ENERGY EXPENDITURE

(71) Applicant: Yale University, New Haven, CT (US)

(72) Inventors: Joseph Schlessinger, New Haven, CT (US); Tamas Horvath, New Haven, CT (US); Mansoor Ahmed, New Haven, CT (US); Navjot Kaur, New Haven, CT (US)

(21) Appl. No.: 18/852,901

(22) PCT Filed: Mar. 24, 2023
(86) PCT No.: PCT/US2023/016203

§ 371 Date: Sep. 30, 2024

Related U.S. Application Data
(60) Provisional application No. 63/324,744, filed on Mar. 29, 2022.

(51) Int. Cl. A61K 38/19; A61K 31/439; A61P 3/08

(52) U.S. Cl. CPC A61K 38/19; A61K 31/439; A61P 3/08

(57) ABSTRACT

Described herein are a method of increasing energy expenditure level in a subject, a method of reducing body weight in a subject, a method of decreasing the amount of white adipose (WAT) tissue and/or promoting browning of WAT in a subject, and/or a method of improving glucose tolerance and/or insulin sensitivity in a subject. Each of the methods includes downregulating the level and/or activity of Augmentor α (Augα), or downregulating the level and/or activity of anaplastic lymphoma kinase (ALK) in the subject. Also described herein are a method of decreasing energy expenditure level in a subject, and/or a method of increasing body weight in a subject. Each of the methods includes upregulating the level and/or activity of Augα, or upregulating the level and/or activity of ALK in the subject.

### CROSS-REFERENCE TO RELATED APPLICATIONS

The present application claims priority under 35 U.S.C. § 119 (e) to U.S. Provisional Patent Application No. 63/324,744, filed Mar. 29, 2022, which is incorporated herein by reference in its entirety.

### STATEMENT REGARDING FEDERALLY SPONSORED RESEARCH

This invention was made with government support under 5R01AG052005-03, 1R01AG067329-01 and 1R01DK126447-01A1 awarded by National Institutes of Health. The government has certain rights in the invention.

### SEQUENCE LISTING

The ASCII text file named “047162-7370WO1 (01945)_Seq Listing.xml” created on Mar. 24, 23023, comprising 4,597 bytes, is hereby incorporated by reference in its entirety.

### BACKGROUND

Increased or decreased energy expenditure, as well as excessively high or excessively low body weight, are implicated in various diseases, disorders, or conditions. For example, obesity corresponds to higher than average body weight and may be caused by certain conditions involving decreased energy expenditure. On the other hand, anorexia nervosa causes dramatic loses in body weight and is often associated with increased energy expenditure.

Therefore, there is a need for compositions and methods that can be used to regulate energy expenditure and/or body weight. The present invention addresses this need.

### SUMMARY

In some aspects, the present invention is directed to the following non-limiting embodiments:

### Method of Increasing Energy Expenditure Levels

In some embodiments, the present invention is directed to a method of increasing energy expenditure levels in a subject in need thereof.

In some embodiments, the method includes administering to the subject an effective amount of at least one selected from the group consisting of: a small molecule inhibitor of Augmentor α (Augα), a protein inhibitor of Augα, a nucleic acid that downregulates Augα level and/or activity by RNA interference, and/or an expression vector expressing the nucleic acid that downregulates Augα level and/or activity by RNA interference, a ribozyme that downregulates Augα level and/or activity, and/or an expression vector expressing the ribozyme, an expression vector including an expression cassette, wherein the expression cassette expresses CRISPR components that downregulate Augα level and/or activity by CRISPR knockout or CRISPR knockdown, and a trans-dominant negative mutant protein of Augα, and/or an expression vector that expresses a trans-dominant negative mutant protein of Augα.

In some embodiments, the method includes administering to the subject an effective amount of at least one selected from the group consisting of: a small molecule inhibitor of anaplastic lymphoma kinase (ALK), a protein inhibitor of ALK, a nucleic acid that downregulates ALK level and/or activity by RNA interference, and/or an expression vector expressing the nucleic acid that downregulates ALK level and/or activity by RNA interference, a ribozyme that downregulates ALK level and/or activity, and/or an expression vector expressing the ribozyme, an expression vector including an expression cassette, wherein the expression cassette expresses CRISPR components that downregulate ALK level and/or activity by CRISPR knockout or CRISPR knockdown, and a trans-dominant negative mutant protein of ALK, and/or an expression vector that expresses a trans-dominant negative mutant protein of ALK.

In some embodiments, the level and/or activity of Augα and/or ALK is downregulated in the brain of the subject.

In some embodiments, the level and/or activity of Augα and/or ALK is downregulated in the hypothalamus of the brain of the subject.

In some embodiments, the method includes administering to the subject a small molecule inhibitor of ALK.

In some embodiments, the small molecule inhibitor of ALK is blood brain barrier-penetrating.

In some embodiments, the small molecule inhibitor of ALK includes Lorlatinib.

In some embodiments, the subject is a mammal.

In some embodiments, the subject is a human.

### Method of Reducing Body Weight

In some aspects, the present invention is directed to a method of reducing body weight in a subject in need thereof.

In some embodiments, the method includes administering to the subject an effective amount of at least one selected from the group consisting of: a small molecule inhibitor of Augα, a protein inhibitor of Augα, a nucleic acid that downregulates the expression level and/or activity of Augα by RNA interference, and/or an expression vector expressing the nucleic acid that downregulates the expression level and/or activity of Augα by RNA interference, a ribozyme that downregulates the expression level and/or activity of Augα, and/or an expression vector expressing the ribozyme, an expression vector including an expression cassette, wherein the expression cassette expresses CRISPR components that downregulate the expression level and/or activity of Augα by CRISPR knockout or CRISPR knockdown, and a trans-dominant negative mutant protein of Augα, and/or an expression vector that expresses the trans-dominant negative mutant protein of Augα.

In some embodiments, the method includes administering to the subject an effective amount of at least one selected from the group consisting of: a small molecule inhibitor of anaplastic lymphoma kinase (ALK), a protein inhibitor of ALK, a nucleic acid that downregulates ALK level and/or activity by RNA interference, and/or an expression vector expressing the nucleic acid that downregulates ALK level and/or activity by RNA interference, a ribozyme that downregulates ALK level and/or activity, and/or an expression vector expressing the ribozyme, an expression vector including an expression cassette, wherein the expression cassette expresses CRISPR components that downregulate ALK level and/or activity by CRISPR knockout or CRISPR knockdown, and a trans-dominant negative mutant protein of ALK, and/or an expression vector that expresses a trans-dominant negative mutant protein of ALK.

In some embodiments, the level and/or activity of Augα and/or ALK is downregulated in the brain of the subject.

In some embodiments, the level and/or activity of Augα and/or ALK is downregulated in the hypothalamus of the brain of the subject.

In some embodiments, the method includes administering to the subject a small molecule inhibitor of ALK.

In some embodiments, the small molecule inhibitor of ALK is blood brain barrier-penetrating.

In some embodiments, the small molecule inhibitor of ALK includes Lorlatinib.

In some embodiments, the subject is a mammal.

In some embodiments, the subject is a human.

### Method of Decreasing the Amount of White Adipose Tissue (WAT) and or Promoting Browning of WAT

In some aspects, the present invention is directed to a method of decreasing the amount of white adipose tissue (WAT) and/or promoting browning of WAT in a subject in need thereof.

In some embodiments, the method includes administering to the subject an effective amount of at least one selected from the group consisting of: a small molecule inhibitor of Augα, a protein inhibitor of Augα, a nucleic acid that downregulates the expression level and/or activity of Augα by RNA interference, and/or an expression vector expressing the nucleic acid that downregulates the expression level and/or activity of Augα by RNA interference, a ribozyme that downregulates the expression level and/or activity of Augα, and/or an expression vector expressing the ribozyme, an expression vector including an expression cassette, wherein the expression cassette expresses CRISPR components that downregulate the expression level and/or activity of Augα by CRISPR knockout or CRISPR knockdown, and a trans-dominant negative mutant protein of Augα, and/or an expression vector that expresses the trans-dominant negative mutant protein of Augα.

In some embodiments, the method includes administering to the subject an effective amount of at least one selected from the group consisting of: a small molecule inhibitor of anaplastic lymphoma kinase (ALK), a protein inhibitor of ALK, a nucleic acid that downregulates ALK level and/or activity by RNA interference, and/or an expression vector expressing the nucleic acid that downregulates ALK level and/or activity by RNA interference, a ribozyme that downregulates ALK level and/or activity, and/or an expression vector expressing the ribozyme, an expression vector including an expression cassette, wherein the expression cassette expresses CRISPR components that downregulate ALK level and/or activity by CRISPR knockout or CRISPR knockdown, and a trans-dominant negative mutant protein of ALK, and/or an expression vector that expresses a trans-dominant negative mutant protein of ALK.

In some embodiments, the level and/or activity of Augα and/or ALK is downregulated in the brain of the subject.

In some embodiments, the level and/or activity of Augα and/or ALK is downregulated in the hypothalamus of the brain of the subject.

In some embodiments, the method includes administering to the subject a small molecule inhibitor of ALK.

In some embodiments, the small molecule inhibitor of ALK is blood brain barrier-penetrating.

In some embodiments, the small molecule inhibitor of ALK includes Lorlatinib.

In some embodiments, the subject is a mammal

In some embodiments, the subject is a human.

### Method of Improving Glucose Tolerance and or Insulin Sensitivity

In some aspects, the present invention is directed to a method of improving glucose tolerance and/or insulin sensitivity in a subject in need thereof.

In some embodiments, the method of improving glucose tolerance and/or insulin sensitivity includes administering to the subject an effective amount of at least one selected from the group consisting of: a small molecule inhibitor of Augα, a protein inhibitor of Augα, a nucleic acid that downregulates the expression level and/or activity of Augα by RNA interference, and/or an expression vector expressing the nucleic acid that downregulates the expression level and/or activity of Augα by RNA interference, a ribozyme that downregulates the expression level and/or activity of Augα, and/or an expression vector expressing the ribozyme, an expression vector including an expression cassette, wherein the expression cassette expresses CRISPR components that downregulate the expression level and/or activity of Augα by CRISPR knockout or CRISPR knockdown, and a trans-dominant negative mutant protein of Augα, and/or an expression vector that expresses the trans-dominant negative mutant protein of Augα.

In some embodiments, the method includes administering to the subject an effective amount of at least one selected from the group consisting of: a small molecule inhibitor of anaplastic lymphoma kinase (ALK), a protein inhibitor of ALK, a nucleic acid that downregulates ALK level and/or activity by RNA interference, and/or an expression vector expressing the nucleic acid that downregulates ALK level and/or activity by RNA interference, a ribozyme that downregulates ALK level and/or activity, and/or an expression vector expressing the ribozyme, an expression vector including an expression cassette, wherein the expression cassette expresses CRISPR components that downregulate ALK level and/or activity by CRISPR knockout or CRISPR knockdown, and a trans-dominant negative mutant protein of ALK, and/or an expression vector that expresses a trans-dominant negative mutant protein of ALK.

In some embodiments, the level and/or activity of Augα and/or ALK is downregulated in the brain of the subject.

In some embodiments, the level and/or activity of Augα and/or ALK is downregulated in the hypothalamus of the brain of the subject.

In some embodiments, the method includes administering to the subject a small molecule inhibitor of ALK.

In some embodiments, the small molecule inhibitor of ALK is blood brain barrier-penetrating.

In some embodiments, the small molecule inhibitor of ALK includes Lorlatinib.

In some embodiments, the subject is a mammal.

In some embodiments, the subject is a human.

Method of decreasing energy expenditure levels in a subject

In some aspects, the present invention is directed to a method of decreasing energy expenditure levels in a subject in need thereof.

In some embodiments, the method includes administering to the subject: an effective amount of at least one selected from the group consisting of Augα, a modified Augα, or a fragment thereof, and/or an expression vector expressing the Augα, modified Augα, or fragment thereof.

In some embodiments, the method includes administering to the subject: an effective amount of at least one selected from the group consisting of ALK, a modified ALK, or a fragment thereof, and/or an expression vector expressing the ALK, modified ALK, or fragment thereof.

In some embodiments, the level and/or activity of Augα and/or ALK is upregulated in the brain of the subject.

In some embodiments, the level and/or activity of Augα and/or ALK is upregulated in the hypothalamus of the brain of the subject.

In some embodiments, the subject is administered with a biologically active fragment of Augα.

In some embodiments, the subject is a mammal.

In some embodiments, the subject is a human.

### Method of Increasing Body Weight

In some aspects, the present invention is directed to a method of increasing body weight in a subject in need thereof,

In some embodiments, the method includes administering to the subject: an effective amount of at least one selected from the group consisting of Augα, a modified Augα, or a fragment thereof, and/or an expression vector expressing the Augα, modified Augα, or fragment thereof.

In some embodiments, the method includes administering to the subject: an effective amount of at least one selected from the group consisting of ALK, a modified ALK, or a fragment thereof, and/or an expression vector expressing the ALK, modified ALK, or fragment thereof.

In some embodiments, the level and/or activity of Augα and/or ALK is upregulated in the brain of the subject.

In some embodiments, the level and/or activity of Augα and/or ALK is upregulated in the hypothalamus of the brain of the subject.

In some embodiments, the subject is administered a biologically active fragment of Augα.

In some embodiments, the subject is a mammal.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIGS. 1A-1I demonstrate that Augα is predominantly expressed in AgRP neurons within the arcuate nucleus and its expression is increased upon fasting, in accordance with some embodiments. FIG. 1A: Expression of Gfp within the hypothalamus in the Augα Gfp/+animals at P90. ARC=Arcuate nucleus, DMH=Dorsomedial nucleus, PVN=Paraventricular nucleus, SCN=Suprachiasmatic nucleus. Scale bar=500 μm. FIG. 1B: Heatmap showing the level of Gfp expression within the Augα-Gfp/+ and Augα-Gfp/Gfp mice at P90 (n=3). FIGS. 1C-1D: Coronal sections of Augα Gfp/+; AgRP-Cre; Ai14-tdT (FIG. 1C) and Augα-Gfp/+; POMC-Cre; Ai14-tdT (FIG. 1D) animals at P60 showing predominant co-localization of Augα and AgRP. Scale bar=40 μm. FIGS. 1E-IF: Expression of GFP (Augα) and RFP (AgRP) within the ARC and PVN of the Augα-Gfp/+; AgRP-Cre; Ai14-tdT and Augα Gfp/Gfp; AgRP-Cre; Ai14-tdT mice under normal conditions (FIG. 1E) and under 16 h of food deprivation (FIG. 1F). Scale bar=40 μm. FIG. 1G: Quantifications of Aug-Gfp expression within the ARC and PVN from FIGS. 1E-1F. Data in the figures is presented as mean±SEM. Two-way ANOVA with Bonferroni's multiple comparisons test is applied; *** p<0.001, ** p<0.01 (n=4). FIG. 1H: Expression of c-Fos within the ARC nuclei of the Augα-Gfp/+; AgRP-Cre; Ai14-tdT and Augα Gfp/Gfp; AgRP-Cre; Ai14-tdT after 16 h of food deprivation. Scale bar=20 μm. FIG. 1I: Quantification of c-Fos in Ai14-tdT positive cells from FIG. 1H. Two-way ANOVA with Bonferroni's multiple comparisons test was applied (n=3).

FIGS. 2A-2J demonstrate that Augα knockout mice are thin, in accordance with some embodiments. FIGS. 2A-2B: Body weight kinetics of Augα+/+; Augα-Gfp/+; Augα-Gfp/Gfp littermate male (FIG. 2A) and female (FIG. 2B) mice fed on standard diet (n≥6). FIG. 2C: Body weight kinetics of Augα-Gfp and Augβ-Gfp single, double knockout and their littermate double heterozygous male mice on normal diet, n≥5. FIGS. 2D-2E. Body weight in the Augα+/+; Augα-Lacz/+; Augα-Lacz/Lacz littermate male (FIG. 2D) and female (FIG. 2E) mice fed on standard diet, A=n≥4; B=n≥5. FIG. 2F: Body weight gain from male and female Augα KO, HET and WT littermate mice on high fat diet, n≥5. FIG. 2G: Representative Image showing the gross morphology of 36-week-old Augα KO, HET and WT mice on standard diet. FIGS. 2H-2J: MRI (magnetic resonance imaging) analysis of fat (FIG. 2H), lean mass (FIG. 2I) and body weight (FIG. 2J), n≥5. Data in the figures is presented as mean±SEM. One or Two-way ANOVA with Bonferroni's multiple comparisons test is applied; * p<0.05, ** p<0.01.

FIGS. 3A-3I demonstrate that Augα knockout mice exhibit decreased adiposity, in accordance with some embodiments. FIG. 3A: Bar plot showing the weights of fat mass from brown (BAT), subcutaneous (scWAT), retroperitoneal (rWAT) and gonadal (gWAT) adipose tissues, n≥8. FIG. 3B: Representative H&E Image of BAT, scWAT, rWAT and gWAT showing decreased size of the adipocytes in Augα KO vs WT. Scale bar=120 μM. FIGS. 3C-3E: Line graph showing quantification of adipocyte area from scWAT, rWAT and gWAT, n=3. FIGS. 3F-3H: qRT-PCR quantification of fatty acid oxidation and thermogenesis genes (UCP1, PCGla, Cidea, Beta3AR) in scWAT, rWAT and gWAT, n≥9. FIG. 3I: Quantification of norepinephrine level from BAT, scWAT, rWAT, gWAT and serum, n≥8. Data in the figure is presented as mean±SEM. Two tailed unpaired student's t test is applied; * p<0.05, ** p<0.01, *** p<0.001, ** p<0.0001.

FIGS. 4A-4I demonstrate that Augα knockout mice showed increased energy expenditure and glucose tolerance, in accordance with some embodiments. FIGS. 4A-4H: Line plots showing the kinetics of (FIG. 4A) Energy expenditure (EE), (FIG. 4B) Respiratory exchange rate (RER), (FIG. 4C) Oxygen consumption (VO₂), (FIG. 4D) CO₂ production (VCO₂), (FIG. 4E) Activity, (FIG. 4F) food intake, and (FIG. 4G) water intake during light (white) and dark (grey) phases, n≥14. FIGS. 4H-4I. Line graph showing kinetics of glucose clearance (FIG. 4H) and insulin tolerance (FIG. 41), n≥12. Data in the figure is presented as mean±SEM. Two tailed unpaired student's t test is applied; * p<0.05, ** p <0.01, *** p<0.001.

FIGS. 5A-5D demonstrate that reduced AgRP projections leads to suppressed Alk phosphorylation in PVN of Augα deficient mice, in accordance with some embodiments. FIG. 5A: Image showing innervation of AgRP fibers within the PVN in Augα-Gfp/Gfp and Augα Gfp/+ mice. FIG. 5B. Quantification of the AgRP innervations within PVN by measuring relative intensity. Data presented as mean±SEM. Two tailed unpaired student's t test is applied; ** p<0.01, n≥6. FIGS. 5C-5D: Images showing the phosphorylated Alk (“pAlk”) within the PVN neurons in Augα-Gfp/+ (FIG. 5C) and Augα-Gfp/Gfp (FIG. 5D) mice brains. The AgRP fibers from the ARC nucleus (“Ai14-tdT”) are in immediate vicinity of Alk expressing cells (arrowheads). The Augα expressing cells are located juxtaposed to the Alk expressing cells (star). Lower panel (scale bar=5 μM) is blow up of dotted box in upper panel (scale bar=20 μM).

FIGS. 6A-6E depicts the results of gene expression analysis of Alk and MC4R positive neurons in PVN, in accordance with some embodiments. FIGS. 6A-6B: Heat map (FIG. 6A) and bar plot (FIG. 6B) showing differentially expressed gene from mouse hypothalamus RNAseq. FIG. 6C: Scattered Plot showing Alk and Crh correlation from the RNAseq. FIG. 6D: Scattered Plot showing Mc4r and Trh correlation from the RNAseq. FIG. 6E: Immunostaining showing colocalization of Alk and Crh in PVN from P50 mice. Lower panel (scale bar=40 μM) is magnified image of dotted box in upper panel (scale bar=200 μM).

FIGS. 7A-7E are schematic of Augα and Augβ knockout mice generation, in accordance with some embodiments. FIG. 7A: Schematic of Augα WT genome locus containing exon1 to exon 4 that were edited using a Gfp expression cassette. Genotyping primers are annotated as P1, P2, P3. FIG. 7B: Schematic of Augβ WT genome locus containing exon1 that was edited using a Gfp expression cassette. Genotyping primers are annotated as P4, P5. FIG. 7C: Gel picture showing the genotyping of Augα and Augβ mice using primer pairs P1+P2, P1+P3 and P4+P5. FIGS. 7D-7E: Schematic of Augα-LacZ knockout mice (FIG. 7D); DNA gel showing genotype of mice by using primer P6+P7 (WT) and P8+P9 (LacZ) (FIG. 7E).

FIGS. 8A-8E show Augα expression in AgRP positive neurons, in accordance with some embodiments. FIGS. 8A-8B: Immunostaining of coronal sections of Augα-gfp/+brains for NPY, and POMC. FIGS. 8C-8D: t-SNE plots of Augα expression in AgRP neurons, from single cell RNAseq data. FIG. 8E: Bar plot showing Augα and Augβ expression in AgRP and POMC neurons during fasting and refeeding state, from single cell RNAseq data.

FIGS. 9A-9B demonstrate that Augα expression is increased upon fasting, in accordance with some embodiments. FIGS. 9A-9B: Expression of GFP (Augα) and RFP (AgRP) within the ARC and PVN of the Augα-Gfp/+; AgRP-Cre; Ai14-tdT and Augα Gfp/Gfp; AgRP-Cre; Ai14-tdT mice under non-fasted condition (FIG. 9A) and 16 h of food deprivation (FIG. 9B). Scale bar=40 μm.

FIG. 10A-10F depict the metabolic dataset of food deprived mice, in accordance with some embodiments. FIGS. 10A-10F: Metabolic parameters; EE (FIG. 10A), RER (FIG. 10B), VO₂ (FIG. 10C), VCO₂ (FIG. 10D), water Intake (FIG. 10E), activity (FIG. 10F). Data presented as mean±SEM, unpaired student's t test is applied; p<0.05, *; ** p<0.01, n≥7.

FIGS. 11A-11G depict the metabolic dataset during refeeding after 16 h fast, in accordance with some embodiments. FIGS. 11A-11D: Bar graph showing cumulative changes in the EE (FIG. 11A), R; ER (FIG. 11B), VO₂ (FIG. 11C), VCO₂ (FIG. 11D) food intake (FIG. 11E), water intake (FIG. 11F) and activity (FIG. 11G) during refeeding after 16 h fasting. Data presented as mean±SEM, unpaired student's t test is applied, p<0.05, *; p<0.01, **; p<0.0001, ****, n≥7.

FIG. 12A-12F shows Augα induced neurodifferentiation, and Alk and Augα localization, in accordance with some embodiments. FIG. 12A: Image showing neurodifferentiation of neuronal stem cells isolated from E18 wild type brain (n=3). Scale bar: 40 μm. FIGS. 12B-12C: In situ hybridizations from Allen brain atlas (FIG. 12B) and in-house (FIG. 12C) showing Alk expression within PVN (portal dot brain-map dot org). Scale bar: 500 μM. FIGS. 12D-12E: Image showing the Alk (“ALK”) immunostaining within the PVN neurons in Augα Gfp/+ and Augα Gfp/Gfp mice brains. Scale bar: 200 μM; 50 μM (zoom in). FIG. 12F: pAlk quantification from FIGS. 5C-5D. Mean integrated intensity of the pAlk stainings within PVN of Augα-Gfp/+ and Augα Gfp/Gfp were calculated (n=4).

FIGS. 13A-13H are plots showing the kinetics and bar plots of summary of various metabolic parameters of control (vehicle treated) animals and animals treated with Lorlatinib or Crizotinib, in accordance with some embodiments. FIG. 13A: food intake, FIG. 13B: water intake, FIG. 13C: energy expenditure, FIG. 13D: oxygen consumption, FIG. 13E: CO₂ production, FIG. 13F: respiratory exchange rate (RER), FIG. 13G: activity, and FIG. 13H: distance traveled in cage during light (white) and dark (grey) phases, n=4/group. Data in the figure is presented as mean±SEM. Unpaired student's t test is applied for P value.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The study described herein (“the present study”) discovered that Augmentor α (Augα), a ligand of the receptor tyrosine kinase ALK, is expressed in the AgRP neurons, which are associated with food intake and energy homeostasis. The present study discovered that downregulating the Augα-ALK pathway (such as the exemplary method of homozygous Augα knockout or pharmaceutical inhibition of ALK) resulted in thinner subjects. The thinner phenotype did not appear to be caused by changes in appetite, but rather was a result of increased energy expenditure. Downregulating the pathway also improved glucose tolerance and insulin sensitivity, as well as decreased white adipose tissue amounts and promoted browning of white adipose tissues.

Accordingly, in some aspects, the present invention is directed to a method of increasing energy expenditure in a subject in need thereof. In some embodiments, the method includes downregulating Augα level and/or activity, or downregulating ALK level and/or activity in the subject, such as in the brain of the subject, such as in the hypothalamus of the brain of the subject.

In some aspects, the present invention is directed to a method of reducing or regulating body weight in a subject in need thereof. In some embodiments, the method includes downregulating Augα level and/or activity, or downregulating ALK level and/or activity in the subject, such as in the brain of the subject, such as in the hypothalamus of the brain of the subject.

In some aspects, the present invention is directed to a method of improving glucose tolerance and/or insulin sensitivity in a subject in need thereof. In some embodiments, the method includes downregulating Augα level and/or activity, or downregulating ALK level and/or activity in the subject, such as in the brain of the subject, such as in the hypothalamus of the brain of the subject.

In some aspects, the present invention is directed to a method of decreasing white adipose tissue (WAT) amount or promoting WAT browning in a subject in need thereof. In some embodiments, the method includes downregulating Augα level and/or activity, or downregulating ALK level and/or activity in the subject, such as in the brain of the subject, such as in the hypothalamus of the brain of the subject.

In some aspects, the present invention is directed to a method of decreasing energy expenditure levels in a subject in need thereof. In some embodiments, the method includes upregulating or stimulating Augα level and/or activity, or upregulating or stimulating ALK level and/or activity in the subject, such as in the brain of the subject, such as in the hypothalamus of the brain of the subject.

In some aspects, the present invention is directed to a method of increasing, preventing further loss of, and/or reversing loss of body weight in a subject in need thereof. In some embodiments, the method includes upregulating Augα level and/or activity, or upregulating ALK level and/or activity in the subject, such as in the brain of the subject, such as in the hypothalamus of the brain of the subject.

In certain aspects, human ALK has the
following amino acid sequence
(SEQ ID NO: 1):
MGAIGLLWLLPLLLSTAAVGSGMGTGQRAGSPAAGPPLQPREPLSYSRLQ
RKSLAVDFVVPSLFRVYARDLLLPPSSSELKAGRPEARGSLALDCAPLLR
LLGPAPGVSWTAGSPAPAEARTLSRVLKGGSVRKLRRAKQLVLELGEEAI
LEGCVGPPGEAAVGLLQFNLSELFSWWIRQGEGRLRIRLMPEKKASEVGR
EGRLSAAIRASQPRLLFQIFGTGHSSLESPTNMPSPSPDYFTWNLTWIMK
DSFPFLSHRSRYGLECSFDFPCELEYSPPLHDLRNQSWSWRRIPSEEASQ
MDLLDGPGAERSKEMPRGSFLLLNTSADSKHTILSPWMRSSSEHCTLAVS
VHRHLQPSGRYIAQLLPHNEAAREILLMPTPGKHGWTVLQGRIGRPDNPE
RVALEYISSGNRSLSAVDFFALKNCSEGTSPGSKMALQSSFTCWNGTVLQ
LGQACDFHQDCAQGEDESQMCRKLPVGFYCNFEDGFCGWTQGTLSPHTPQ
WQVRTLKDARFQDHQDHALLLSTTDVPASESATVISATFPAPIKSSPCEL
RMSWLIRGVLRGNVSLVLVENKTGKEQGRMVWHVAAYEGLSLWQWMVLPL
LDVSDRFWLQMVAWWGQGSRAIVAFDNISISLDCYLTISGEDKILQNTAP
KSRNLFERNPNKELKPGENSPRQTPIFDPTVHWLFTTCGASGPHGPTQAQ
CNNAYQNSNLSVEVGSEGPLKGIQIWKVPATDTYSISGYGAAGGKGGKNT
MMRSHGVSVLGIFNLEKDDMLYILVGQQGEDACPSTNQLIQKVCIGENNV
IEEEIRVNRSVHEWAGGGGGGGGATYVFKMKDGVPVPLIIAAGGGGRAYG
AKTDTFHPERLENNSSVLGLNGNSGAAGGGGGWNDNTSLLWAGKSLQEGA
TGGHSCPQAMKKWGWETRGGFGGGGGGCSSGGGGGGYIGGNAASNNDPEM
DGEDGVSFISPLGILYTPALKVMEGHGEVNIKHYLNCSHCEVDECHMDPE
SHKVICFCDHGTVLAEDGVSCIVSPTPEPHLPLSLILSVVTSALVAALVL
AFSGIMIVYRRKHQELQAMQMELQSPEYKLSKLRTSTIMTDYNPNYCFAG
KTSSISDLKEVPRKNITLIRGLGHGAFGEVYEGQVSGMPNDPSPLQVAVK
TLPEVCSEQDELDFLMEALIISKFNHQNIVRCIGVSLQSLPRFILLELMA
GGDLKSFLRETRPRPSQPSSLAMLDLLHVARDIACGCQYLEENHFIHRDI
AARNCLLTCPGPGRVAKIGDFGMARDIYRASYYRKGGCAMLPVKWMPPEA
FMEGIFTSKTDTWSFGVLLWEIFSLGYMPYPSKSNQEVLEFVTSGGRMDP
PKNCPGPVYRIMTQCWQHQPEDRPNFAIILERIEYCTQDPDVINTALPIE
YGPLVEEEEKVPVRPKDPEGVPPLLVSQQAKREEERSPAAPPPLPTTSSG
KAAKKPTAAEISVRVPRGPAVEGGHVNMAFSQSNPPSELHKVHGSRNKPT
SLWNPTYGSWFTEKPTKKNNPIAKKEPHDRGNLGLEGSCTVPPNVATGRL
PGASLLLEPSSLTANMKEVPLFRLRHFPCGNVNYGYQQQGLPLEAATAPG
AGHYEDTILKSKNSMNQPGP

Residues 19-1038 correspond to the extracellular domain. Residues 1039-1059 correspond to the helical transmembrane domain. Residues 1060-1620 correspond to the cytoplasmic domain.

In certain aspects, human Auga has the
following amino acid sequence
(SEQ ID NO: 2):
MRGPGHPLLLGLLLVLGAAGRGRGGAEPREPADGQALLRLVVELVQELRK
HHSAEHKGLQLLGRDCALGRAEAAGLGPSPEQRVEIVPRDLRMKDKFLKH
LTGPLYFSPKCSKHFHRLYHNTRDCTIPAYYKRCARLLTRLAVSPVCMED
KQ

wherein the underlined residues correspond to the signal peptide, the bold residues correspond to the variation region, and the plain/regular font correspond to the AUG domain.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments±5%, in certain embodiments±1%, in certain embodiments±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intraperitoneal, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

As used herein, the term “energy expenditure” refers to the amount of energy a subject uses to maintain essential body functions (respiration, circulation, digestion) and as a result of physical activity. Total daily energy expenditure is determined by resting or basal metabolic rate (BMR), food-induced thermogenesis, and energy expended as a result of physical activity. As used herein, the term “basal metabolic rate” or “BMR” refers to the minimum amount of energy that the body of an endothermic animal subject requires for essential organ and cellular function when lying in a state of physiological and mental rest. In some embodiments, the methods described herein change the level of energy expenditure other than the basal metabolic rate portion.

As used herein, the term “glucose tolerance” means the ability of a subject to dispose of a glucose load. As used herein, the term “insulin sensitivity” or “sensitivity to insulin” describes how sensitive the body is to the effects of insulin on glucose disposal.

As used herein, the terms “downregulating a/the level” or “upregulating a/the level” of a protein or a gene refer to increasing or decreasing the amount of the protein or gene. The levels of a protein or a gene can be downregulated by, for example, decreasing the copy number of the gene, decreasing the expression level of the protein/gene, or increasing the rate of removal or degradation of the protein or mRNAs producing the protein. Similarly, the levels of a protein or a gene can be upregulated by, for example, increasing the copy numbers of the gene, increasing the expression level of the protein/gene, or decreasing the rate of removal or degradation of the protein or mRNAs producing the protein. As used herein, the terms “downregulating an activity” or “upregulating an activity” of a protein or a gene refer to increasing or decreasing the ability of the protein or gene to carry out its normal functions at a given amount. The activities of a protein or gene can be downregulated by, for example, contacting with an inhibitor or an antagonist. The activities of a protein or gene can be downregulated by, for example, contacting with an activator or an agonist.

Abbreviation: ARC: arcuate nucleus. PVN: paraventricular nucleus. DMH: dorsomedial nucleus. SCN: suprachiasmatic nucleus. EE: energy expenditure. RER: respiratory exchange rate.

### Method of Increasing Energy Expenditure

Reduced energy expenditure is implicated in various diseases, disorders, or conditions. For example, reduced energy expenditure plays an important role in the development of at least some types of obesity (Fonseca et al., Clinical Nutrition Experimental Volume 20, August 2018, Pages 55-59) and several obesity related comorbities (Khaodhiar et al. Clin Cornerstone. 1999; 2 (3): 17-31). Certain medications, such as long term use of some beta blockers (see e.g., Lamont, J Cardiopulm Rehabil. May-June 1995; 15 (3): 183-5.), are known to cause reduced energy expenditure, as well. The present study discovered that downregulating Augα-ALK pathway (such as decreasing Augα and/or ALK level) resulted in increased energy expenditure level.

Accordingly, in some aspects, the present invention is directed to a method of increasing energy expenditure in a subject in need thereof.

In some embodiments, the method includes downregulating Augα level and/or activity, or downregulating ALK level and/or activity in the subject.

In some embodiments, the level and/or activity of Augα and/or ALK is downregulated in the brain of the subject, such as in the hypothalamus of the brain of the subject.

In certain embodiments, the level of Augα and/or ALK comprises the expression level of Augα and/or ALK.

In some embodiments, downregulating the level and/or the activity of Augα includes administering to the subject an effective amount of:
a small molecule inhibitor of Augα,
a protein inhibitor of Augα,
a nucleic acid that downregulates the level and/or activity of Augα by RNA interference, and/or an expression vector expressing the nucleic acid that downregulates the level and/or activity of Augα by RNA interference,
a ribozyme that downregulates the level and/or activity of Augα, and/or an expression vector expressing the ribozyme, an expression vector comprising an expression cassette, wherein the expression cassette expresses CRISPR components that downregulate the level and/or activity of Augα by CRISPR knockout or CRISPR knockdown,
a trans-dominant negative mutant protein of Augα, and/or an expression vector that expresses the trans-dominant negative mutant protein of Augα, or
combinations thereof.

In some embodiments, downregulating the level and/or activity of ALK includes administering to the subject an effective amount of:
a small molecule inhibitor of ALK,
a protein inhibitor of ALK,
a nucleic acid that downregulates the level and/or activity of ALK by RNA interference, and/or an expression vector expressing the nucleic acid that downregulates the level and/or activity of ALK by RNA interference,
a ribozyme that downregulates the level and/or activity of ALK, and/or an expression vector expressing the ribozyme,
an expression vector comprising an expression cassette, wherein the expression cassette expresses CRISPR components that downregulate the level and/or activity of ALK by CRISPR knockout or CRISPR knockdown,
a trans-dominant negative mutant protein of ALK, and/or an expression vector that expresses the trans-dominant negative mutant protein of ALK, or
combinations thereof.

What is considered as “effective amount” by the specification is described elsewhere herein.

Downregulating Augα and/or ALK with Small Molecule Inhibitors

In some embodiments, the compound that downregulates the level and/or activity of Augα includes a small molecule that inhibits the activity of Augα and/or ALK. As used herein, the term “small molecule” refers to a molecule having a size of less than 2000, 1800, 1600, 1400, 1200, 1000, 800, or 600 daltons.

In some embodiments, the small molecule inhibitor comprises a PROTAC or a Proteolysis Targeting Chimeric Molecule. PROTACs are heterobifunctional nanomolecules that can target any protein for ubiquitination and degradation. In certain embodiments, the PROTAC contemplated in the present invention comprises a group that is recognized by the E3 ubiquitin ligase and a group that is recognized by Augα and/or ALK. The PROTAC is able to simultaneously bind to the Augα and/or ALK and the E3 ligase. Formation of such trimeric complex formation leads to the transfer of ubiquitins to the Augα and/or ALK, marking it for degradation. PROTAC molecules possess good tissue distribution and the ability to target intracellular proteins, thus can be directly applied to cells or injected into animals without the use of vectors. PROTACS useful within the invention can be prepared using any known compound that binds to and/or recognizes and/or inhibits Augα and/or ALK, which is linked through a linker to an E3 ubiquitin ligase, such as but not limited to those described in WO 2013/106643, WO 2013/106646, and WO 2019/148055.

Furthermore, three generations of small molecule ALK inhibitors have been developed to treat ALK positive cancers, such as ALK positive non-small cell lung cancer (NSCLC). All of these small molecule ALK inhibitors are expected to work for the methods herein. Some of the small molecule ALK inhibitors are able to penetrate blood brain barrier (BBB) and can be used directly. Other small molecule ALK inhibitors are less effective at penetrating BBB. For these small molecule ALK inhibitors, brain specific drug delivery routes or BBB-penetrating carriers are sometimes needed or preferred. BBB-penetrating carriers are described in, for example, Pinheiro et al. (Int J Mol Sci. 2021 November; 22 (21): 11654) and Ahlawat et al. (ACS Omega 2020, 5, 22, 12583-12595). The brain specific drug delivery routes are described elsewhere herein. Examples of small molecule ALK inhibitors include Alectinib, Alkotinib (also known as ZG-0418), AP26113, ASP3026, AZD3463, Belizatinib (also known as TSR-011), Brigatinib, CEP-28122, CEP-37440, Certinib, Crizotinib, Ensartinib (also known as X-396), Entrectinib (also known as NMS-E628 and RXDX-101), Foritinib (SAF-189), HG-14-10-04, Lorlatinib, PF-06463922, PLB1003, Repotrectinib (also known as TPX-0005), TAE684, TPX-0131, TQ-B3139, TSR-011, X-376, or derivatives thereof. ALK inhibitors are well known and three generations of ALK inhibitors are publicly available. As such, one of ordinary skill in the art would be able to select suitable compounds to inhibit ALK for the purposes of the instant methods.

Downregulating Augα with Protein Inhibitors of Augα

In some embodiments, the compound that downregulates the level and/or activity of Augα and/or ALK includes a protein that downregulates the level and/or activity of Augα and/or ALK.

In some embodiments, the protein that downregulates the level and/or activity of Augα and/or ALK includes antibodies, non-antibody proteins, and/or combinations thereof.

Non-limiting examples of monoclonal and/or polyclonal antibodies that target Augα include bs-8219R by Bioss, NBP1-90646 by Novus Biologicals, orb2217 by Biorbyt, PA5-55591 by Thermo Fisher Scientific, and any humanized derivatives thereof.

Non-limiting examples of monoclonal and/or polyclonal antibodies that target ALK include those made by Moog-Lutz et al. (J Biol Chem. 2005 Jul. 15; 280 (28): 26039-48), 4C5B8 by Invitrogen, UM800118 by OriGene, and any humanized derivatives thereof.

One of ordinary skill in the art would expect that extracellular fragments of ALK would be able to sequester Augα and other ligands of ALK, thereby acting as non-antibody protein inhibitors of Augα and/or ALK.

In some embodiments, the protein that downregulates the level and/or activity of Augα and/or ALK is administered in form of a protein. In some embodiments, the protein that downregulates the level and/or activity of Augα and/or ALK is administered in form of a nucleic acid that expresses the protein, such as an expression vector. The expression vector is described in the “Vector” section elsewhere in the instant specification.

Downregulating Augα and/or ALK by RNA Interference

In some embodiments, the compound that downregulates the activity and/or level of Augα and/or ALK includes a nucleic acid that downregulates the activity and/or level of Augα and/or ALK by the means of RNA interference.

In some embodiments, the nucleic acid that downregulates the level of Augα and/or ALK by the means of RNA interference includes an isolated nucleic acid. In other embodiments, the modulator is an RNAi molecule (such as but not limited to siRNA and/or shRNA and/or miRNAs) or antisense molecule, which inhibits the expression and/or activity of Augα and/or ALK. In yet other embodiments, the nucleic acid comprises a promoter/regulatory sequence, such that the nucleic acid is preferably capable of directing expression of the nucleic acid. Thus, the instant specification provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of Augα and/or ALK. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391 (19): 306-311; Timmons et al., 1998, Nature 395:854; Montgomery et al., 1998, TIG 14 (7): 255-258; Engelke, Ed., RNA Interference (RNAi) Nuts & Bolts of RNAi Technology, DNA Press, Eagleville, PA (2003); and Hannon, Ed., RNAi A Guide to Gene Silencing, Cold Spring Harbor Laboratory Press, Cold Spring Harbor, NY (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the instant specification also includes methods of decreasing levels of Augα and/or ALK using RNAi technology.

In certain embodiments, the instant specification provides a vector comprising an siRNA or antisense polynucleotide. In other embodiments, the siRNA or antisense polynucleotide inhibits the expression of Augα and/or ALK. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide has certain characteristics that can be modified to improve the siRNA as a therapeutic compound. Therefore, in some embodiments, the siRNA polynucleotide is further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

In some embodiments, the RNA interference oligonucleotides are specifically designed to increase the cellular uptake of these oligonucleotides. Methods of designing oligonucleotides having desirable cellular uptake are described in, e.g., Geary et al., Adv Drug Deliv Rev 87, 46-51 (2015) and Crooke et al., Nature biotechnology 35, 230-237 (2017).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine. In some embodiments, the oligonucleotides exist as cholesterol conjugated DNA/RNA heteroduplex oligonucleotides (HDOs) such that the oligonucleotides are blood-brain barrier permeable and could reach the central nervous system (CNS) after subcutaneous or intravenous administration (Nagata et al., Nature biotechnology (2021)).

In certain embodiments, an antisense nucleic acid sequence expressed by a plasmid vector is used to inhibit Augα and/or ALK protein expression. The antisense expressing vector is used to transfect a mammalian cell or the mammal itself, thereby causing reduced endogenous expression of Augα and/or ALK.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the instant specification may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the instant specification include oligonucleotide derivatives known in the art which have improved biological activity compared to unmodified oligonucleotides (see U.S. Pat. No. 5,023,243).

Downregulating Augα and/or ALK with a Ribozyme

In some embodiments, the compound that down regulates the activity or level of Augα and/or ALK includes a ribosome that inhibits Augα and/or ALK protein expression.

A ribozyme is used to inhibit Augα and/or ALK protein expression. Ribozymes useful for inhibiting the expression of a target molecule may be designed by incorporating target sequences into the basic ribozyme structure which are complementary, for example, to the mRNA sequence encoding Augα and/or ALK. Ribozymes are antisense RNAs which have a catalytic site capable of specifically cleaving complementary RNAs. Therefore, ribozymes having sequence complementary to Augα and/or ALK mRNA sequences are capable of downregulating the expression of Augα and/or ALK by reduces the level of Augα and/or ALK mRNA. Ribozymes targeting Augα and/or ALK may be synthesized using commercially available reagents (Applied Biosystems, Inc., Foster City, CA) or they may be genetically expressed from DNA encoding them. In some embodiments, the DNA encoding the ribozymes are incorporated in a vector, which is described in the “Vector” section elsewhere in the instant specification.

Downregulating Augα and/or ALK by CRISPR Knockout/Knockdown and Other Knockouts/Knockdown Techniques

In some embodiments, the compound that downregulates the activity or level of Augα and/or ALK comprises a nucleic acid that down regulates the level of Augα and/or ALK by the means of CRISPR knockout.

In some embodiments, the compound downregulates the activity or level of Augα and/or ALK comprises a CRISPR/Cas9 system for knocking out Augα and/or ALK.

The CRISPR/Cas9 system is a facile and efficient system for inducing targeted genetic alterations. Target recognition by the Cas9 protein requires a “seed” sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The RecI domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US2014/0068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to neurons, stem cells and immune cells (Addgene, Cambridge, MA, Mirus Bio LLC, Madison, WI).

The guide RNA is specific for a genomic region of interest and targets that region for Cas endonuclease-induced double strand breaks. The target sequence of the guide RNA sequence may be within a locus of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the instant specification. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the instant specification may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, the compound that down regulates the activity or level of Augα and/or ALK comprises a nucleic acid that down regulates the level of Augα and/or ALK by the means of CRISPR knockdown. CRISPR knockdown includes, but not limited to, CRISPRCas13 knockdown. (See e.g., Mendez-Mancilla et al., Cell Chemical Biology 29, 1-7, 2021 Jul. 27, and Kushawah et al., Dev Cell. 2020 Sep. 28; 54 (6): 805-817. The entireties of which are incorporated herein by reference)

In some embodiments, the present invention includes any other methods for effecting gene knockdown and/editing, which allow for deletion and/or inactivation of Augα and/or ALK such as but not limited to those described in WO 2018/236840 (which is incorporated herein in its entirety by reference).

Downregulating Augα and/or ALK by Inactivating and/or Sequestering

In some embodiments, the compound that downregulates the activity or level of Augα and/or ALK includes a protein that downregulates the activity of Augα and/or ALK by inactivating and/or sequestering Augα and/or ALK. In some embodiments, the compound includes a nucleic acid that express the protein that downregulates the activity of Augα and/or ALK by inactivating and/or sequestering Augα and/or ALK. In some embodiments, the compound includes an expression vector that express the protein that downregulates the activity of Augα and/or ALK by inactivating and/or sequestering Augα and/or ALK (see “Vector” section for descriptions on vectors).

In some embodiments, the compound that downregulates the level of Augα and/or ALK is a trans-dominant negative mutant of Augα and/or ALK, and/or a nucleic acid or a vector expressing the trans-dominant negative mutant of Augα and/or ALK.

### Method of Reducing Body Weight

Excessively high body weight is implicated in various diseases, disorders, or conditions, such as obesity and type 2 diabetes. Medications used to treat many psychiatric conditions such as depression or schizophrenia are also known to cause increased body weight (see e.g., Shrivastava et al., Mens Sana Monogr. 2010 January-Dec; 8 (1): 53-68). The present study discovered that downregulating Augα-ALK pathway (such as decreasing the levels of Augα or decreasing the activities of ALK) resulted in decreased body weight.

Accordingly, in some aspects, the present invention is directed to a method of reducing body weight in a subject in need thereof.

In some embodiments, the method includes downregulating level and/or activity of Augα or downregulating ALK level and/or activity in the subject.

In some embodiments, the level and/or the activity of Augα and/or ALK is downregulated in the brain of the subject, such as in the hypothalamus of the brain of the subject.

In some embodiments, the level and/or the activity of Augα and/or ALK is downregulated in the same or similar manners as those described elsewhere herein, such as in the “Method of Increasing Energy Expenditure” section.

Method of Improving Glucose Tolerance and/or Insulin Sensitivity

Reduced glucose tolerance and/or insulin sensitivity is implicated in various diseases, disorders, or conditions. For example, subjects having prediabetes and diabetes have lower than normal glucose tolerance and insulin resistance. Glucose intolerance and insulin resistance have also been associated with propagation of neurodegenerative disorders, such as Alzheimer's disease (see e.g., Cai et al., Current Alzheimer Research, Volume 9, Number 1, 2012, pp. 5-17 (13)). The present study discovered that downregulating Augα-ALK pathway (such as by decreasing the levels of Auga) is able to improve glucose tolerance and increase insulin sensitivity.

Accordingly, in some aspects, the present invention is directed to a method of improving glucose tolerance and/or insulin sensitivity in a subject in need thereof.

In some embodiments, the method includes downregulating Augα level and/or activity, or downregulating ALK level and/or activity in the subject.

In some embodiments, the level and/or activity of Augα and/or ALK is downregulated in the brain of the subject, such as in the hypothalamus of the brain of the subject.

In some embodiments, the level and/or the activity of Augα and/or ALK is downregulated in the brain of the subject, such as in the hypothalamus of the brain of the subject.

In some embodiments, the level and/or the activity of Augα and/or ALK is downregulated in the same or similar manners as those described elsewhere herein, such as in the “Method of Increasing Energy Expenditure” section.

### Method of Decreasing White Adipose Tissue (WAT) Amounts or Promoting Browning of WAT

Excessive white adipose tissue amounts in the bodies of subjects or whitening of brown adipose tissues are implicated in various diseases, disorders, or conditions, such as obesity (e.g., adult obesity and childhood obesity) and diabetes. The present study discovered that downregulating Augα-ALK pathway (such as by decreasing the levels of Auga) is able to decrease the amount of white adipose tissues (WATs) and promote the browning of WATs.

Accordingly, in some aspects, the present invention is directed to a method of decreasing white adipose tissue (WAT) amounts or promoting browning of WAT in a subject in need thereof.

In some embodiments, the method includes downregulating the Level and/or activity of Augα and/or ALK in the subject.

In some embodiments, the level and/or the activity of Augα and/or ALK is downregulated in the brain of the subject, such as in the hypothalamus of the brain of the subject.

In some embodiments, the level and/or the activity of Augα and/or ALK is downregulated in the same or similar manners as those described elsewhere herein, such as in the “Method of Increasing Energy Expenditure” section.

### Method of Decreasing Energy Expenditure Level

Higher than normal energy expenditure level is implicated in certain diseases, disorders or conditions. For example, anorexia nervosa patients expend more energy as physical activities (Casper et al., June 1991 American Journal of Clinical Nutrition 53 (5): 1143-50). Cachexia involves higher than normal energy expenditure and increased baseline energy expenditure is a hallmark of cancer cachexia (see e.g., Dhanapal et al., J Oral Maxillofac Pathol. 2011 September-Dec; 15 (3): 257-260). The present study discovered that downregulating Augα-ALK pathway (such as by decreasing the levels of Augα or decreasing the activities of ALK) is able to increase energy expenditure level. Thus, it is logical that increasing the level and/or activity of Augα and/or ALK would be able to induce the opposite effects.

Accordingly, in some aspects, the present invention is directed to a method of decreasing energy expenditure in a subject in need thereof.

In some embodiments, the method includes upregulating the Level and/or activity of Augα and/or ALK in the subject.

In some embodiments, the level and/or activity of Augα and/or ALK is upregulated in the brain of the subject, such as in the hypothalamus of the brain of the subject.

In some embodiments, the level and/or the activity of Augα is upregulated by administering to the subject an effective amount of Augα, modified Augα, or fragments thereof. Examples of modified Auga/fragments of Augα include the biologically active Augα fragments as described in Reshetnyak et al. (Proc. Natl Acad. Sci. USA 115, 8340-8345 (2018)). In some embodiments, the Augα fragment, the modified Augα, or the fragment of the modified Augα has higher binding affinity toward ALK in comparison to wildtype Augα.

In some embodiments, the level and/or the activity of ALK is upregulated by administering to the subject an effective amount of ALK, a modified ALK, or fragments thereof.

In some embodiments, the Augα, modified Augα, ALK, modified ALK, or any fragment thereof, is administered as a protein, or a nucleic acid (such as an mRNA or a DNA) encoding the protein. In some embodiments, a nucleic acid encoding the Augα, modified Augα, ALK, modified ALK or any fragment thereof, is cloned onto an expression vector or is a part of an expression cassette. Expression vectors are described elsewhere herein.

In some embodiments, the Augα, modified Augα, ALK, modified ALK, or any fragment thereof is delivered to the brain directly. In some embodiments, the Augα, modified AugαALK, modified ALK, or any fragment thereof is delivered to the brain using a blood-brain-barrier penetrating carrier. Both the delivery routes and the BBB-penetrating carriers are described elsewhere herein.

### Method of Increasing Body Weight

Excessively low body weight is implicated in certain diseases, disorders or conditions. For example, anorexia nervosa often involves lower than 85% of expected body weight. Diseases involving wasting, such as cachexia, such as cancer cachexia, cause excessively low body weight, as well (see e.g., Dhanapal et al., J Oral Maxillofac Pathol. 2011 September-Dec; 15 (3): 257-260). The present study discovered that downregulating Augα-ALK pathway (such as by decreasing the levels of Augα or decreasing the activities of ALK) is able to decrease body weight. Thus, it is logical that increasing the level and/or activity of Augα and/or ALK would be able to induce the opposite effects.

Accordingly, in some aspects, the present invention is directed to a method of increasing body weight in a subject in need thereof.

In some embodiments, the method includes upregulating Augα level and/or activity, or upregulating ALK level and/or activity in the subject.

In some embodiments, the level and/or the activity of Augα and/or ALK is upregulated in the brain of the subject, such as in the hypothalamus of the brain of the subject.

In some embodiments, the level and/or the activity of Augα and/or ALK is upregulated in the same or similar manners as those described elsewhere herein, such as in the “Method of Decreasing Energy Expenditure Level” section.

### Pharmaceutical Composition

In some embodiments, the present invention is directed to a pharmaceutical composition.

In some embodiments, the pharmaceutical composition is a composition for increasing energy expenditure in a subject in need thereof. In some embodiments, the pharmaceutical composition is a composition for reducing body weight in a subject in need thereof. In some embodiments, the composition is a composition for improving glucose tolerance and/or insulin sensitivity in a subject in need thereof. In some embodiments, the composition is a composition for decreasing white adipose tissue (WAT) amounts or promoting browning of WAT in a subject in need thereof. In some embodiments, the pharmaceutical composition comprises a compound for downregulating the level and/or activity of Augα and/or ALK (such as the compounds described elsewhere herein, such as in the “Method of Increasing Energy Expenditure” section), and at least one pharmaceutically acceptable carrier.

In some embodiments, the pharmaceutical composition is a composition for decreasing energy expenditure level in a subject in need thereof. In some embodiments, the pharmaceutical composition is a composition for increasing body weight. In some embodiments, the pharmaceutical composition comprises a compound for upregulating the level and/or activity of Augα and/or ALK (such as the compounds described elsewhere herein, such as in the “Method of Decreasing Energy Expenditure Level” section), and at least one pharmaceutically acceptable carrier.

### Vectors

Vectors can increase the stability of the nucleic acids, make the delivery easier, or allow the expression of the nucleic acids or protein products thereof in the cells.

Therefore, in some embodiments, the protein inhibitors or the nucleic acids that modulates the activity or expression level of Augα and/or ALK is incorporated into a vector.

In some embodiments, the instant specification relates to a vector, including the nucleic acid sequence of the instant specification or the construct of the instant specification. The choice of the vector will depend on the host cell in which it is to be subsequently introduced. In certain embodiments, the vector of the instant specification is an expression vector. Suitable host cells include a wide variety of prokaryotic and eukaryotic host cells. In certain embodiments, the expression vector is selected from the group consisting of a viral vector, a bacterial vector and a mammalian cell vector. Prokaryote- and/or eukaryote-vector based systems can be employed for use with the instant specification to produce polynucleotide, or their cognate polypeptides. Many such systems are commercially and widely available.

In some embodiments, the vector is a viral vector. Viral vector technology is well known in the art and is described, for example, in virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In some embodiments, the viral vector is a suitable adeno-associated virus (AAV), such as the AAV1-AAV8 family of adeno-associated viruses. In some embodiments, the viral vector is a viral vector that can infect a human. The desired nucleic acid sequence, such as the nucleic acids that modulate Augα and/or ALK described above, can be inserted between the inverted terminal repeats (ITRs) in the AAV. In various embodiments, the viral vector is an AAV2 or an AAV8. The promoter can be a thyroxine binding globulin (TBG) promoter. In various embodiments, the promoter is a human promoter sequence that enables the desired nucleic acid expression in the brain. In some embodiments, the promoter is a neuron-selective promoter or a neuron-specific promoter. The AAV can be a recombinant AAV, in which the capsid comes from one AAV serotype and the ITRs come from another AAV serotype. In various embodiments, the AAV capsid is selected from the group consisting of a AAV1, AAV2, AAV3, AAV4, AAV5, AAV6, AAV7, and a AAV8 capsid. In various embodiments, the ITR in the AAV is at least one ITR selected from the group consisting of a AAV1, AAV2, AAV3, AAV4, AAV5, AAV6, AAV7, and an AAV8 ITR. In various embodiments, the instant specification contemplates an AAV8 viral vector (recombinant or non-recombinant) containing a desired nucleic acid expression sequence and at least one promoter sequence that, when administered to a subject, causes elevated systemic expression of the desired nucleic acid. In some embodiments, the viral vector is a recombinant or non-recombinant AAV2 or AAV5 containing any of the desired nucleic acid expression sequences described herein. In some embodiments, the AAV is an engineered AAVs for delivering nucleic acid across the blood brain barrier to the central and peripheral nervous systems, such as those as described by Chan et al. (Nat Neurosci. 2017 August; 20 (8): 1172-1179). The entirety of this reference is incorporated herein by reference.

In some embodiments, the vector in which the nucleic acid sequence is introduced is a plasmid that is or is not integrated in the genome of a host cell when it is introduced in the cell. Illustrative, non-limiting examples of vectors in which the nucleotide sequence of the instant specification or the gene construct of the instant specification can be inserted include a tet-on inducible vector for expression in eukaryote cells.

The vector may be obtained by conventional methods known by persons skilled in the art (Sambrook et al., 2012). In certain embodiments, the vector is a vector useful for transforming animal cells.

In certain embodiments, the recombinant expression vectors may also contain nucleic acid molecules which encode a peptide or peptidomimetic inhibitor of the instant specification, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

### Combination Therapies

In some embodiments, in addition to the compound(s) for downregulating or upregulating Augα and/or ALK, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating energy expenditure or body weight related conditions, and/or at least one additional agent that treats one or more diseases or disorders contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of energy expenditure or body weight related conditions in a patient.

Formulations may be employed in admixtures with conventional excipients, i.e., pharmaceutically acceptable organic or inorganic carrier substances suitable for intracranially, intrathecal, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. patents application Ser. Nos. 20/030,147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of the energy expenditure or body weight related condition in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%,25%,30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### Delivery

In certain embodiments, the compound contemplated herein (including but not limited to nucleic acids) can be more efficiently delivered to the cell nucleus by coupling the compound with the monoclonal anti-DNA antibody 3E10, which penetrates living cells and localizes in the nucleus without causing any apparent harm to the cell (Hansen J E, et al., Intranuclear protein transduction through a nucleoside salvage pathway. J Biol Chem 2007; 282:20790-3; see also WO 2020/047353 and WO 2021/042060, all of which are incorporated herein in their entireties by reference). 3E10 and its single-chain variable fragment (3E10 scFv) have been developed as an intracellular delivery system for macromolecules. After localizing in the cell nucleus, 3E10 scFv is largely degraded within 4 hours, thus further minimizing any potential toxicity.

In certain embodiments, the compounds contemplated herein (including but not limited to nucleic acids) can be more efficiently delivered to the central nervous system using certain lipid nanoparticle formulations known in the art, such as but not limited to those described in Cullis, P. R. et al., Molecular Therapy Vol. 25 No 7 Jul. 2017. See also US20150165039 and WO 2014/008334, all of which are incorporated herein in their entireties by reference.

In certain embodiments, the compounds contemplated herein can be more efficiently delivered to tissue by coupling with certain protein fragments, called “pHLIP” (pH (Low) Insertion Peptide), which allow for the cargo to accumulate in acidic environments within the body. In certain embodiments, a polypeptide with a predominantly hydrophobic sequence long enough to span a membrane lipid bilayer as a transmembrane helix (TM) and comprising one or more dissociable groups inserts across a membrane spontaneously in a pH-dependent fashion placing one terminus inside cell. The polypeptide conjugated with various functional moieties delivers and accumulates them at cell membrane with low extracellular pH. The functional moiety conjugated with polypeptide terminus placed inside cell are translocated through the cell membrane in cytosol. The peptide and its variants or non-peptide analogs can be used to deliver therapeutic, prophylactic, diagnostic, imaging, gene regulation, cell regulation, or immunologic agents to or inside of cells in vitro or in vivo in tissue at low extracellular pH. See also US20080233107, WO2012/021790, US20120039990, US20120142042, US20150051153, US20150086617, and US20150191508, all of which are incorporated herein in their entireties by reference.

### Examples

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

Augmentor α and B (AugαAugβ) are ligands of the receptor tyrosine kinases Alk and Ltk. Augα functions as a dimeric ligand that binds with high affinity and specificity to Alk and Ltk. However, a monomeric Augα fragment and monomeric Augβ also bind to Alk and potently stimulate cellular responses. While previous studies demonstrated that oncogenic Alk mutants function as important drivers of a variety of human cancers, the physiological roles of Augα and Augβ are poorly understood. Here the present study investigated the physiological roles of Augα and Augβ by exploring mice deficient in each or both Aug ligands. Analysis of mutant mice shows that both Augα single knockout, and double knockout of Augα and Augβ exhibit similar thinness phenotype and resistance to diet-induced obesity without change in food intake. By contrast, Augβ knockout mice showed no weight loss in comparison to littermate control. Experiments are presented demonstrating that Augα is robustly expressed and metabolically regulated in AgRP neurons, cells that control whole-body energy homeostasis in part, via their projections to the paraventricular nucleus (PVN). Moreover, both Alk and Melanocortin receptor-4 (MC4R) are expressed in discrete neuronal population in PVN and are regulated by projections containing Augα and AgRP, respectively demonstrating that two distinct mechanisms that regulate pigmentation operate in the hypothalamus to control body weight. These experiments show that Alk driven cancers were co-opted from a neuronal pathway in central control of body weight, offering new therapeutic opportunities for metabolic diseases and cancer.

The receptor tyrosine kinase ALK was originally discovered as an oncogenic fusion protein generated in anaplastic large cell lymphoma. A variety of oncogenic ALK fusion proteins were subsequently identified as key drivers of subsets of different cancers including non-small cell lung cancer patients, large B-cell lymphomas and inflammatory myofibroblast tumors. In addition, activating oncogenic somatic mutations were identified in populations of pediatric neuroblastoma patients. Crizotininb, Lorlatinib and other drugs that inhibit the tyrosine kinase activity of ALK were successfully applied for treatment of patients harboring oncogenic ALK mutants. The present study presents experiments demonstrating that the physiological ligands of ALK function in hypothalamus to control body weight offering new therapeutic treatments for metabolic diseases and cancer.

### Example 2

Receptor tyrosine kinases (RTKs) represent an important family of cell surface receptors that regulate numerous essential cellular responses during embryonic development and in homeostasis of adult organism. Alk was originally discovered as an oncogenic RTK fusion protein endowed with constitutively activated tyrosine kinase activity generated by chromosomal translocation in a subset of anaplastic large cell lymphoma. At least 20 distinct partners of oncogenic Alk-fusion proteins generated by chromosomal translocations as well as germline and somatic Alk mutations were identified as key drivers of a variety of cancers including large B-cell lymphomas, inflammatory myofibroblast tumors and in pediatric neuroblastoma. As physiological ligands of full-size Alk and of a second family member designated Ltk were not known for two decades, the two RTKs were classified as “orphan receptors”.

In 2014, Zhang et al. (Proc Natl Acad Sci USA 111, 15741-15745 (2014)) identified two secreted proteins of unknown function designated FAM150A and FAM150B as ligands that bind specifically to and stimulate Ltk activation. It was subsequently demonstrated that these proteins, also designated Augmentor α (Augα) or ALKAL2 and Augmentor β (Augβ) or ALKAL1, function as specific and potent ligands of Alk. Biochemical characterization showed that Augα functions as a dimeric ligand of both Alk and Ltk and that a conserved cysteine residue located in the N-terminal variable region of primate Augα is responsible for mediating Augα dimerization via formation of a disulfide bond between two Augα molecules. A monomeric fragment composed of the conserved C terminal region of Augα stimulates efficiently activation and cell signaling in Alk or Ltk expressing cultured cells. Recent biochemical and structural characterization demonstrated that Augβ functions as a monomeric activating ligand of Alk and Ltk demonstrating and revealing a mechanism of how dimeric or monomeric Aug proteins stimulate Alk dimerization, activation and cellular signaling.

The first insight concerning the biological role of Augmentor proteins emerged from genetic studies of Zebrafish pigment development. These studies demonstrated that Zebrafish Aug-homologs and Alk-homologs are components of a cellular signaling pathway controlling neural crest-derived pigment cells development in Zebrafish It also provided in vivo evidence for a functional ligand/receptor link between Aug and Alk homologs during neural crest development in Zebrafish.

### Example 3: Augα is Expressed in AgRP-Positive Neurons within the Arcuate Nucleus in Hypothalamus

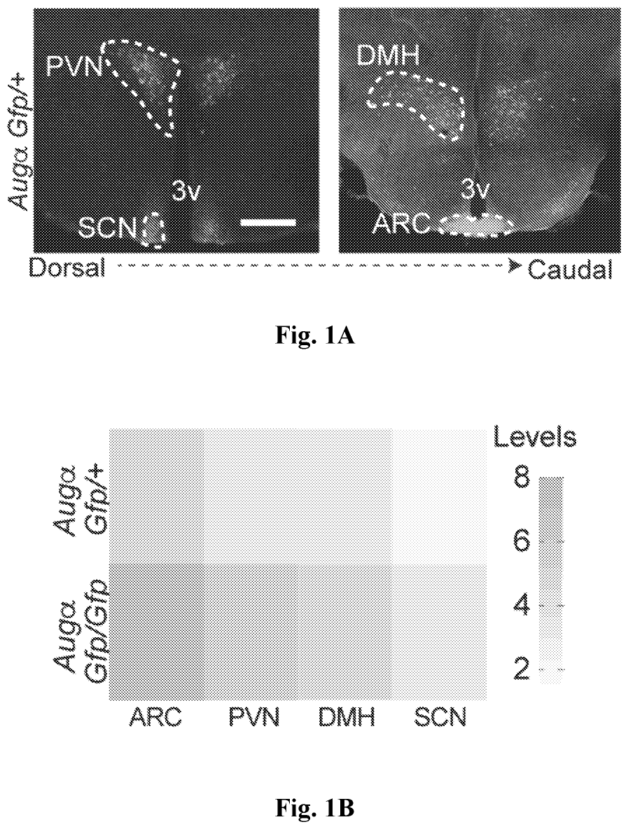

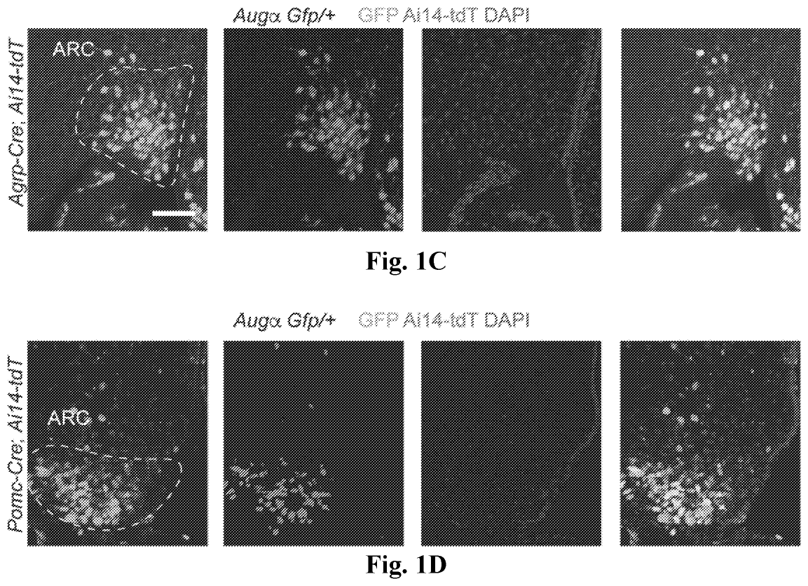

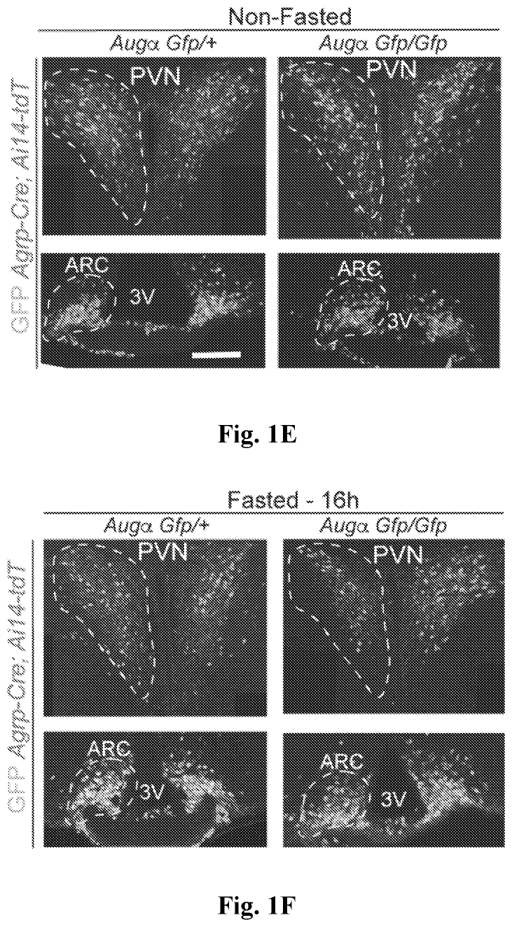

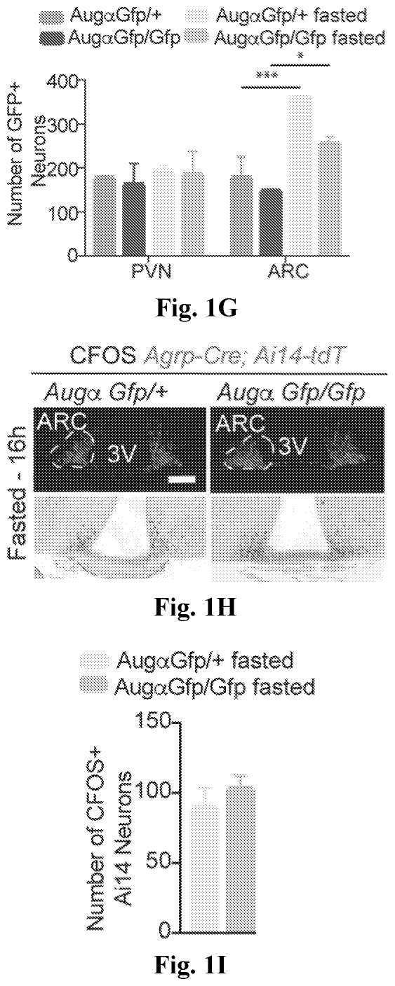

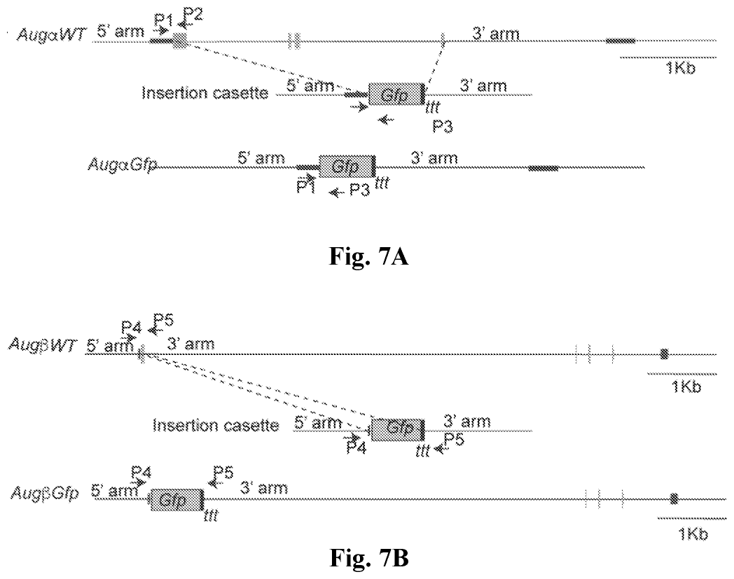

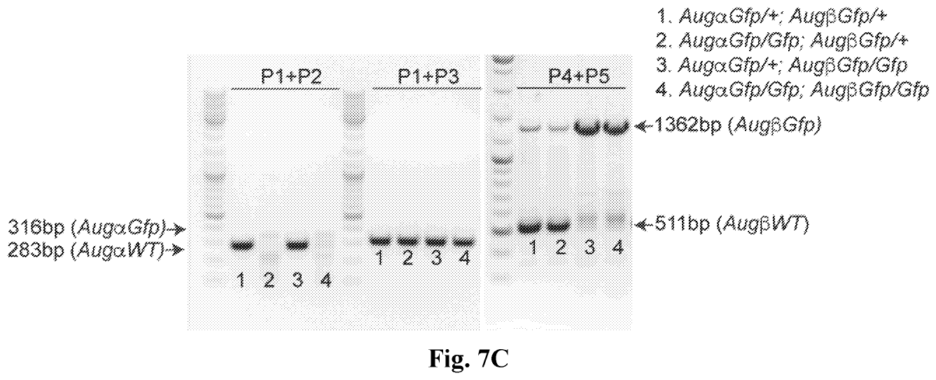

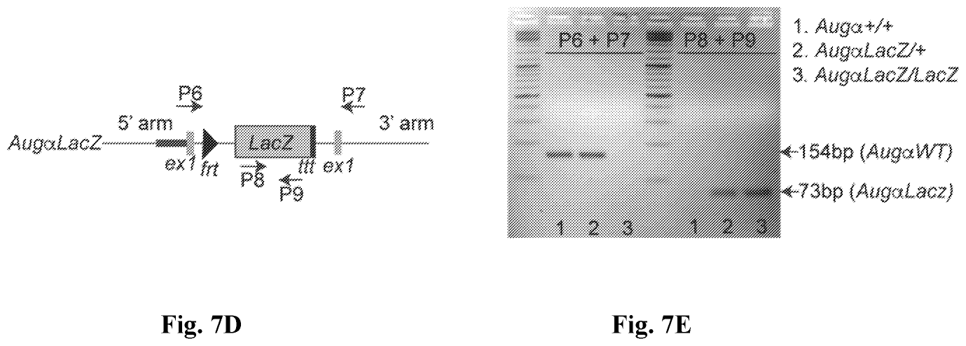

To elucidate the physiological roles of Augα and Augβ in mammals, the present study generated individual knockout mice and double knockout mice of Augα and Augβ. Augα knockout mice were generated by replacing exon 1 to 4 with a GFP expression cassette to enable analysis of the expression pattern of endogenous Augα protein in both tissues and cells. Similarly, Augβ knockout mice were generated by replacing exon 1 with a GFP expression cassette. Double knockout mice were generated by crossing Augα and Augβ deficient mice (FIGS. 1A-1C and 7A-7E). The present study also generated another line of knockout mice by replacing Augα with a LacZ cassette, i.e., AugαLacZ/+ and AugαLacZ/LacZ mice (FIGS. 7D-7E).

Using Augα-Gfp/+ and Augβ-Gfp/+ mice, the present study next analyzed the expression of Augα and Augβ primarily within the brain. Visualization of Augα Gfp/+ mice by immunofluorescence microscopy revealed strong expression in the hypothalamus (FIG. 1A). To determine whether loss of Augα may cause changes in cytoarchitecture and in the localization of neuronal cell populations expressing Augα, the present study generated and similarly analyzed Auga-Gfp/Gfp knockout mice. No obvious defects were observed in the overall brain anatomy and in the region containing cells labeled by Augα-GFP (FIG. 1A). The most robust expression of Augα was detected within the arcuate nucleus (ARC), with a weaker expression in the paraventricular nucleus (PVN), dorsomedial nucleus (DMH), and suprachiasmatic nucleus (SCN) (FIGS. 1A-1B).

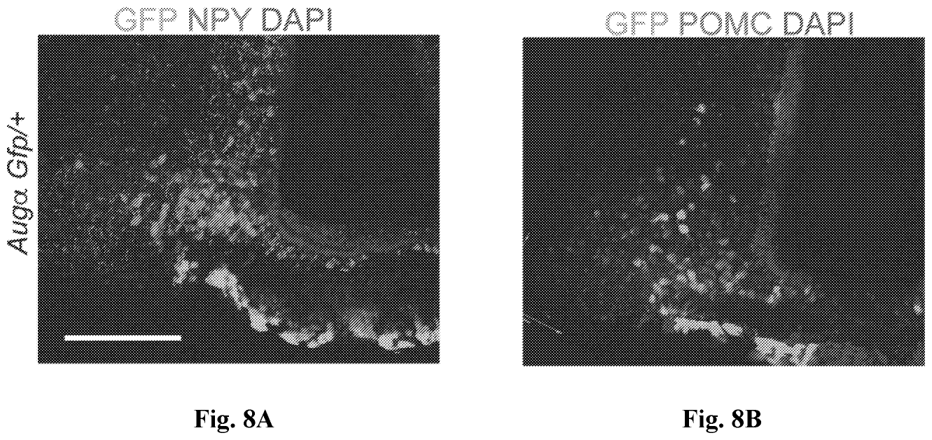

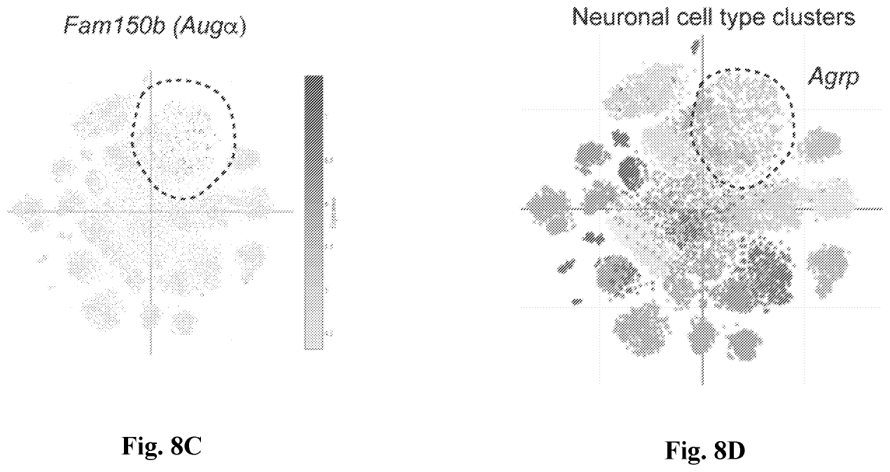

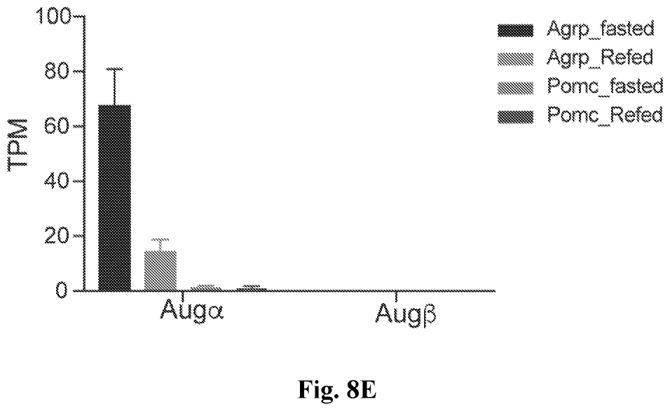

Next, the present study determined the cell types expressing Augα within the ARC nucleus. The ARC nucleus contains two major neuronal cell populations that control energy metabolism, including hunger promoting AgRP neurons and satiety promoting POMC neurons. To determine the specific cells within ARC that express Augα, the present study immunolabelled P60 (postnatal day 60) coronal sections of Augα-Gfp/+ mice with anti-NPY (neuro-peptide Y) antibody, which also labels AgRP neurons (FIG. 8A) and with an anti-POMC antibody that labels POMC neurons (FIG. 8B). This experiment showed increased co-expression of NPY-positive neurons with GFP labeled neurons, suggesting endogenous expression of Augα in AgRP neurons within ARC. Moreover, single-cell RNA-sequencing data revealed high expression of Augα (Fam150B) within AgRP neurons amongst 18 neuronal cell populations (FIG. 8C-8D). To further establish co-localization of Augα-Gfp within the ARC neuronal populations, the present study genetically labeled Augα-Gfp expressing cells within the ARC nucleus using AgRP-Cre; Ail4-tdT mice that label all AgRP expressing neurons (“Ai14-tdT”); and POMC-Cre; Ail4-tdT mice that label all POMC expressing neurons (“Ai14-tdT”) (FIGS. 1C-1D). The Augα-Gfp neurons within the ARC showed strong co-localization with the AgRP expressing neurons and minimal co-localization with POMC neurons. The present study was not able to detect Augβ expression in the brain, which is consistent with open-label RNA-seq data, which also did not reveal Augβ mRNA expression in the brain (FIG. 8E). Taken together these experiments show a particularly high expression of Augα in AgRP neurons in the ARC nucleus of the hypothalamus.

### Example 4: Fasting Stimulates Augα Expression in AgRP Neurons

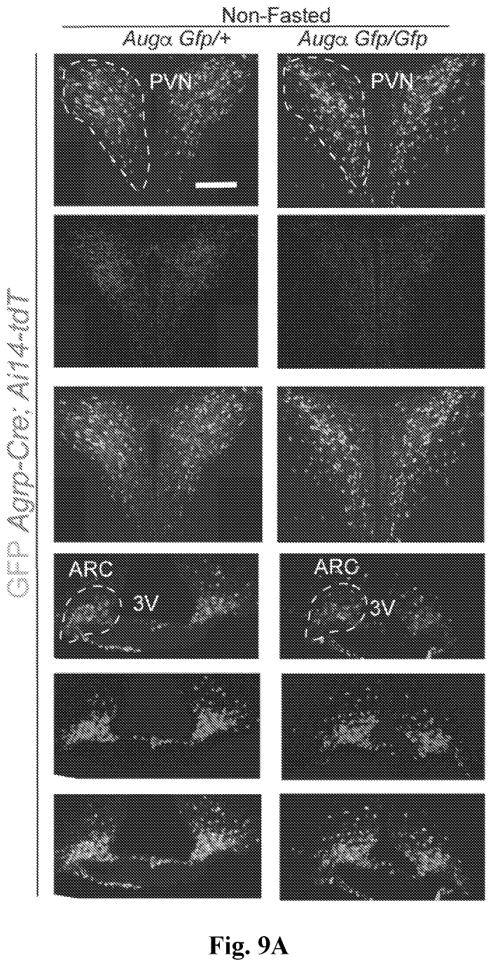

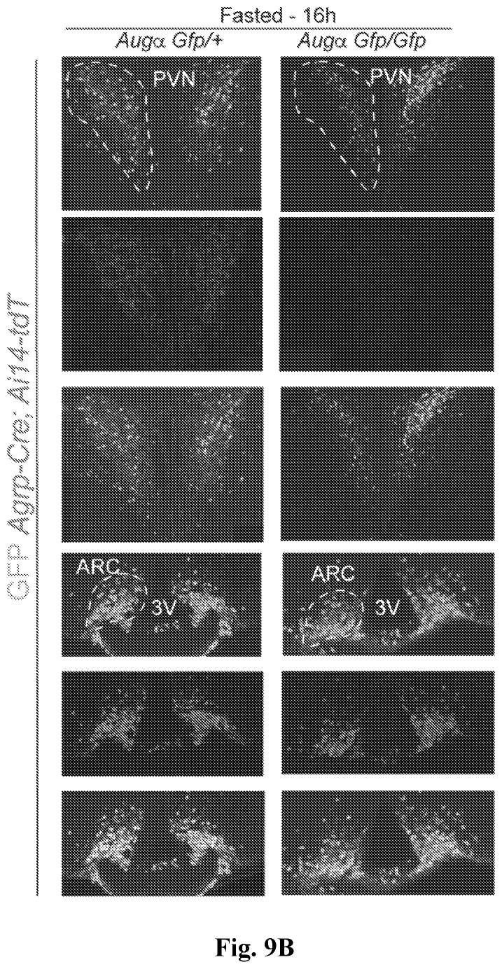

The AgRP neurons located within ARC of the hypothalamus are critical for regulating food intake and energy homeostasis. In response to fasting, AgRP neurons show increased expression of various orexigenic molecules including, AgRP peptide, which regulates neurons in PVN. To determine whether Augα expression within the ARC nucleus is involved in control of hunger and satiety, the present study performed a 16 h food starvation on the Augα-Gfp/+; AgRP-Cre; Ai14-tdT and Augα-Gfp/Gfp; AgRP-Cre; Ai14-tdT animals. It was observed that Augα is metabolically stimulated upon fasting within AgRP neurons while its expression within PVN remains unchanged (FIGS. 1E-1G and 9A-9B). RNAseq data also showed similarly increased expression of Augα in AgRP neurons upon fasting vs refeeding (FIG. 8E). However, the increased expression of Augα did not affect AgRP neurons activation as shown by c-Fos immunostaining in Augα-Gfp/Gfp Vs Augα-Gfp/+animals (FIGS. 1H-1I), suggesting that Augα may act downstream from AgRP signaling.

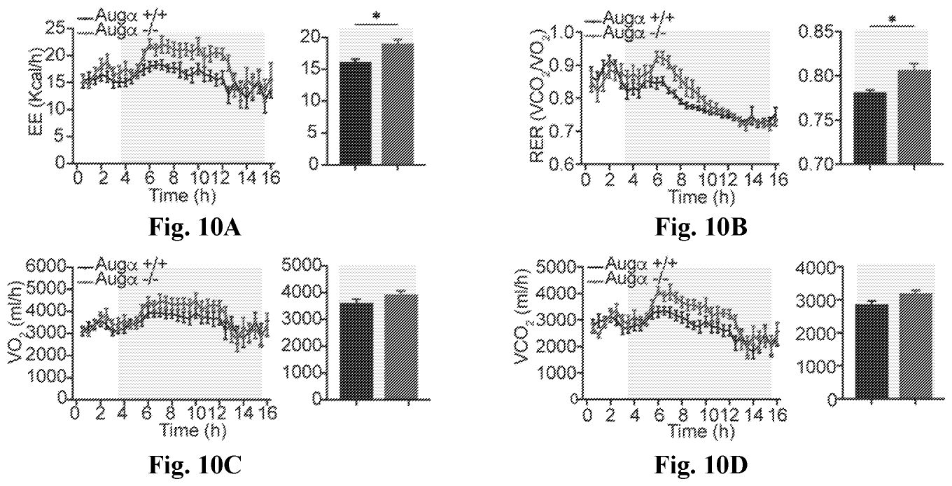

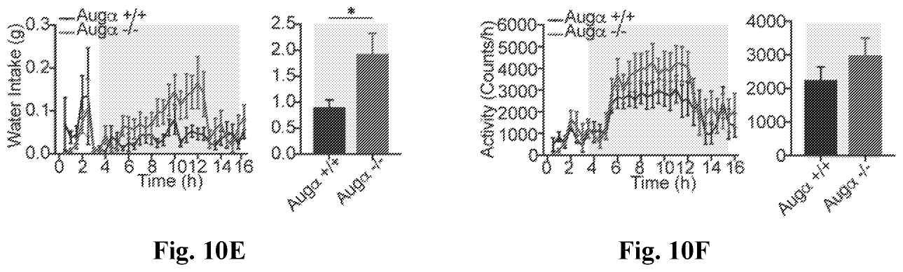

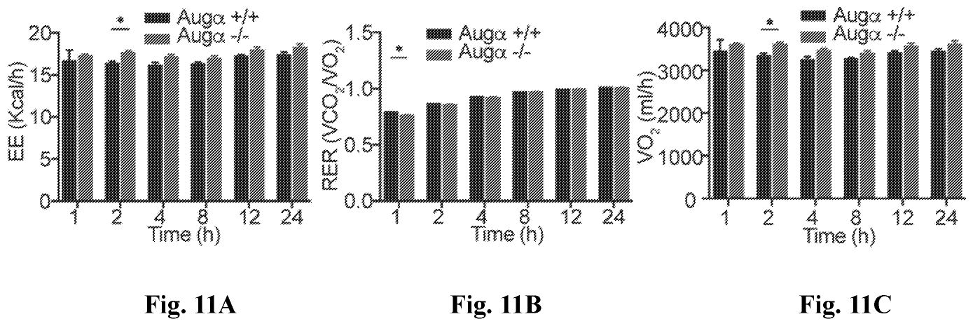

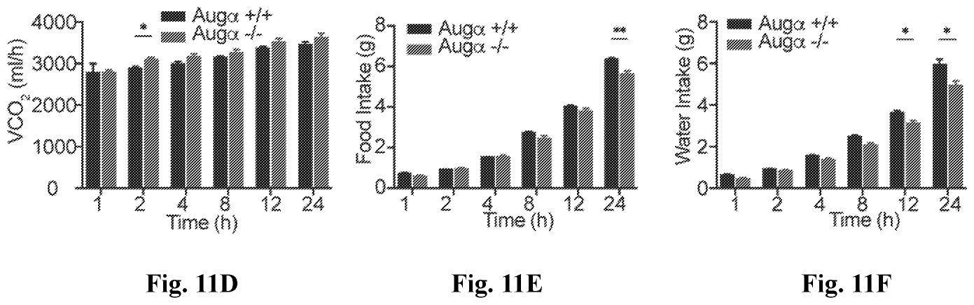

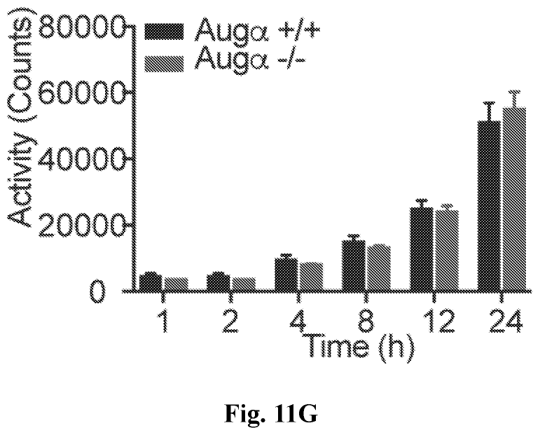

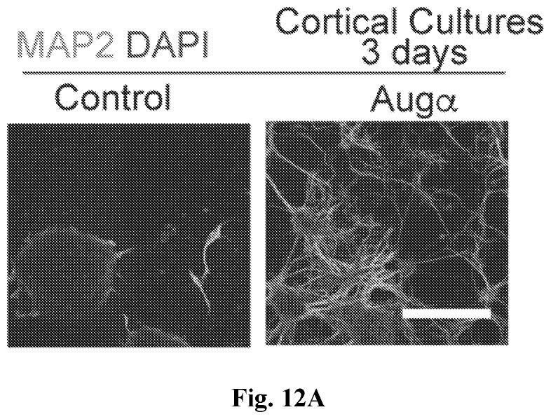

To determine whether Augα governs any of the metabolic phenotype during fasting, the present study performed fasting and refeeding experiments on Augα knockout and wild-type littermate mice in metabolic cages. Single animals were housed for four days in the metabolic cage, and at the end of day four, the mice were food deprived for 16 h and then allowed to feed for the next 24 h. During food deprivation, a significantly higher energy expenditure (EE), and respiratory exchange rate (RER) of Augα deficient mice was detected due to increased CO₂ production during the dark phases indicating a preference for carbohydrates consumption over lipids with activity trending higher (FIGS. 10A-10E). A significant increase in water intake was also detected in Augα deficient mice during the dark phase of fasting period (FIG. 10E). However, upon refeeding, a small but significant decrease in both food and water intake was observed between Augα deficient mice and their wild-type littermates (FIGS. 11A-11E). Taken together, the results showed that Augα expression increases upon fasting similar to the expression of other orexigenic genes, including AgRP and Npy. These results link Augα to the metabolic circuit of AgRP controlled energy homeostasis.

### Example 5: Augα Knockout Mice are Thin

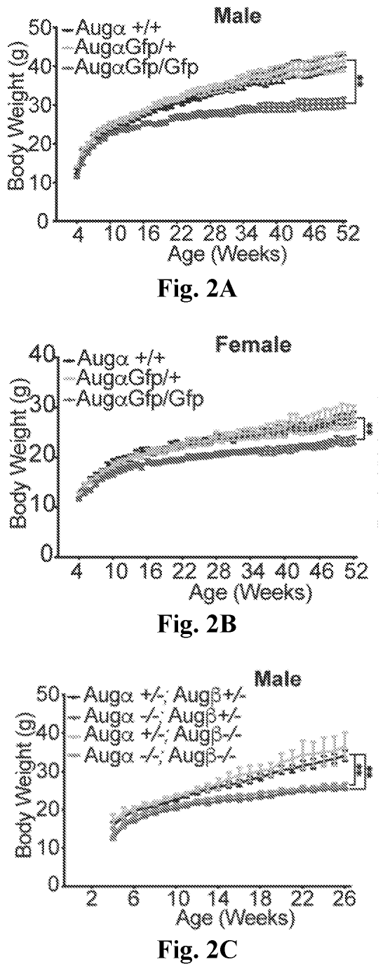

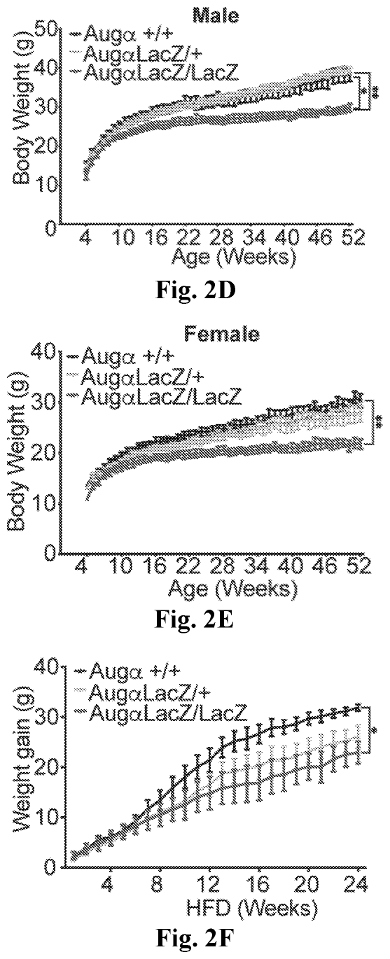

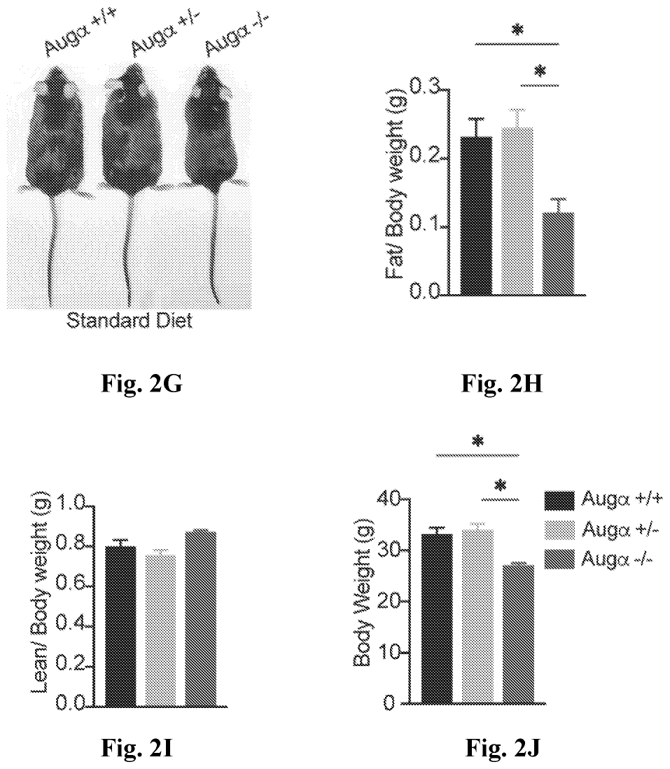

To determine the effect of Augα deficiency on animal physiology, the present study assessed the weight and body composition of the Augα knockout and littermate control mice. The mice were born according to the Mendelian ratio. The present study observed that both Augα male and female knockout mice, derived from both Gfp and LacZ knockin mouse lines, gained less weight with age than wild-type littermate or heterozygous mice on standard chow diet. Differences in body weights were noticed from the age of four weeks onwards. Moreover, from the age of 14 weeks onward, Augα knockout animals (male and female) were significantly leaner compared to Augα heterozygous or wild-type animals. There was no significant weight difference between heterozygous and wild-type mice (FIG. 2A-2B and 2D-E). The Augα and Augβ double knockout mice and Augα single knockout mice showed similar age-dependent weight differences compared to the double heterozygous mice. In contrast, the Augβ knockout mice, which carry one Augα allele, showed no weight difference compared to double heterozygous littermates. These results demonstrate that Augα deficiency is critical for thinness, while Augβ deficiency does not cause thinness (FIG. 2C). In addition, Augα knockout mice gained significantly less weight compared to littermate control when the mice were fed high-fat diet (HFD), suggesting that Augα deficiency provides resistance against HFD induced weight gain (FIG. 2F). Inspection of the gross morphology of the animals revealed a substantial reduction in the circumference of the animals, but no change in their length, indicating that the differences seen in the weights of males and females are not due to change in body length (FIG. 2G). The present study next analyzed the body composition of adult Augα knockout mice and their littermate wild-type as well as heterozygous controls by magnetic resonance imaging (MRI). This analysis revealed that Augα knockout mice have significantly lesser overall body fat and increased lean mass in comparison to wild-type and heterozygous littermate mice, with respect to their overall body weight (FIG. 2H-2J).

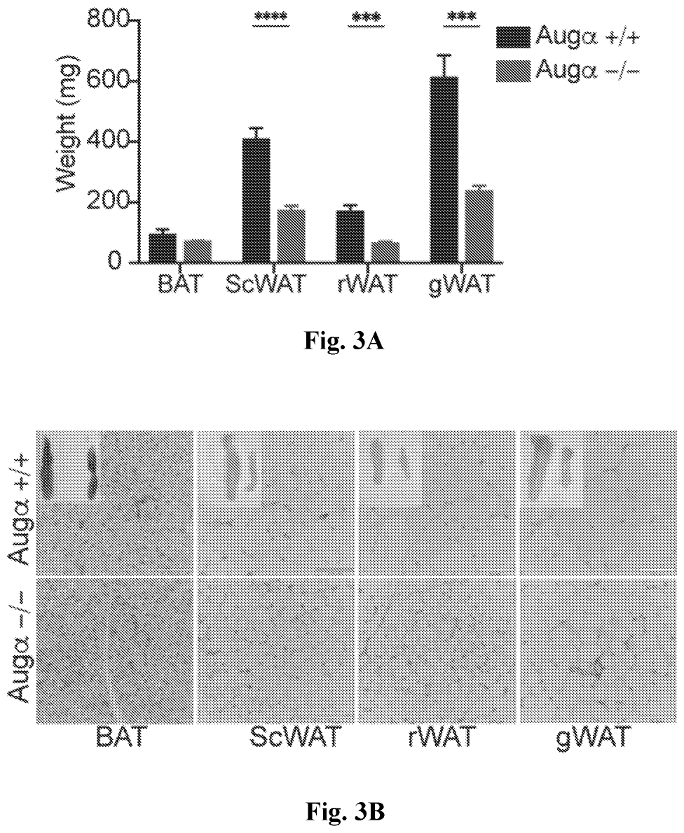

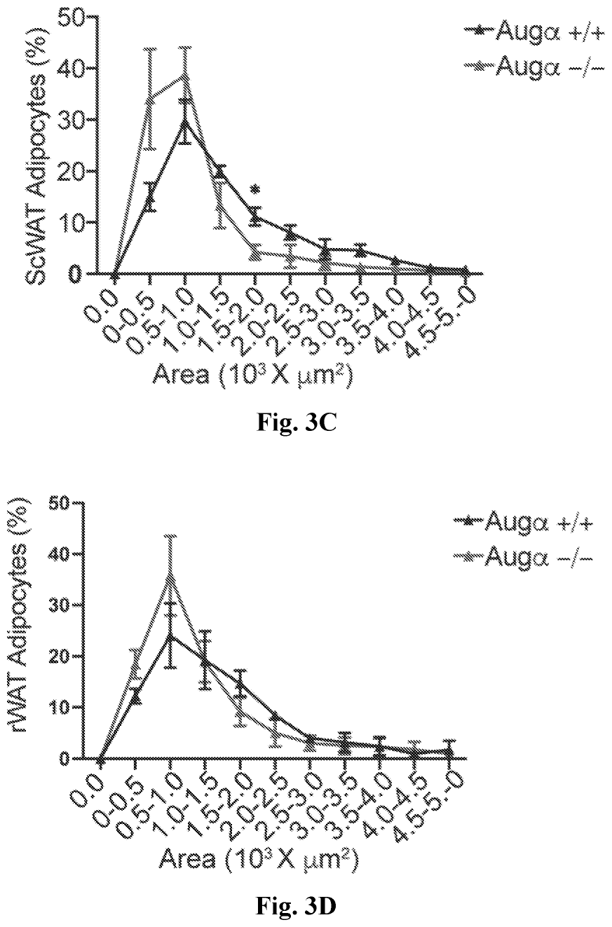

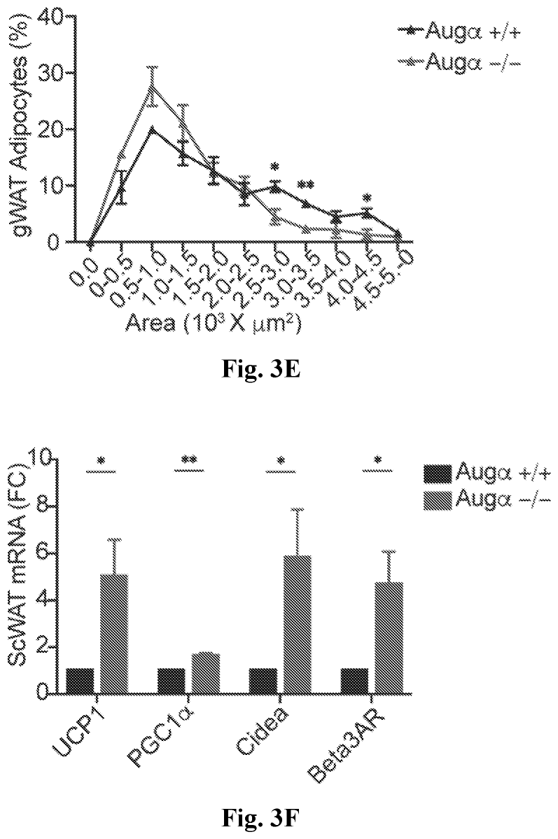

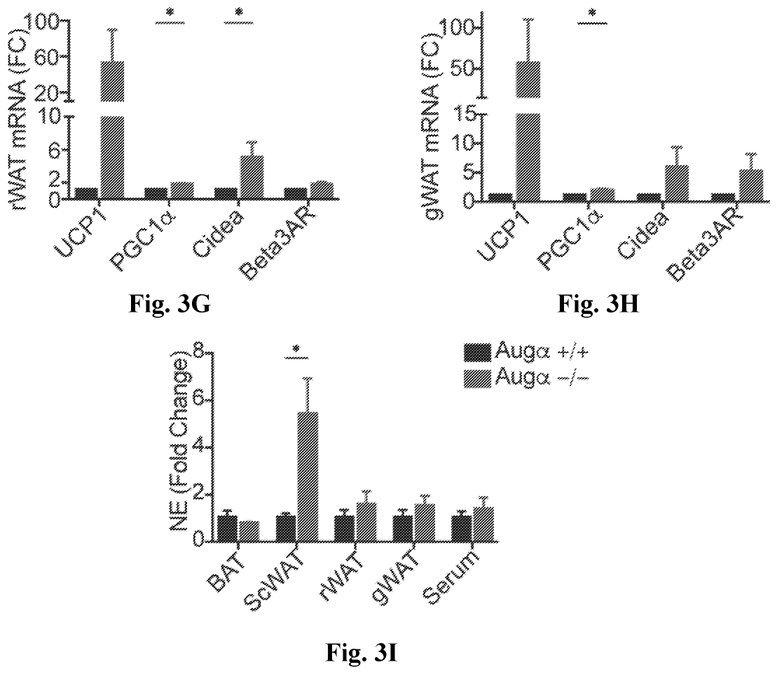

Furthermore, to determine the contributions of each of the fat deposits, fat from subcutaneous (scWAT), retroperitoneal (rWAT) and gonadal white adipose tissue (gWAT) as well as brown adipose 189 tissue (BAT) from Augα knockouts and the wild-type animals were isolated. The results presented in FIG. 3A revealed significantly reduced weight of the scWAT, rWAT and gWAT in Augα knockout mice. Overall gross anatomy revealed reduction in the size of the adipose tissue depots in the Augα knockout mice, which were further analyzed by hematoxylin and eosin (H&E) staining to assess mice's fat composition and adiposity. Overall, lower percentage of large adipocytes were detected in Augα knockout mice compared to their littermate controls, conversely a higher percentage of small adipocyte were detected in Augα knockout mice (FIG. 3B-E). To gain insight into the mechanism of reduced fat deposition in the adipocytes of Augα knockout mice, quantitative real-time PCR (qRT-PCR) analysis was carried out for genes involved in control of thermogenesis and fat oxidation. The experiment presented in FIGS. 3F-3H reveals increase in expression levels of mitochondrial brown fat uncoupling protein 1 (UCP1), peroxisome proliferator-activated receptor co-activator 1α (PGC1α), cell death activator (Cidea-A) and B-3 adrenergic receptor (B3AR) in white adipose tissue (WAT) depots, indicating an increased browning of WAT. To determine a potential link between decreased Augα expression in brain and increased browning of WAT, the present study next analyzed the level of norepinephrine (NE) produced in adipose tissue and in serum of Augα knockout and control mice. This experiment revealed (FIG. 31) a strong increase of NE levels in ScWAT. It was previously demonstrated that hypothalamic PVN neurons regulate NE production within adipocytes through a cascade of neuronal connections resulting in enhancement of thermogenesis and fat oxidation. The results suggest that increased thermogenesis of WAT in Augα knockout mice is controlled by the sympathetic tone of the nervous system which results in reduced fat accumulation, leading to overall reduced weight as the mice age.

### Example 6: Increased Energy Expenditure and Activity Leads to Thinness of Augα Knockout Mice

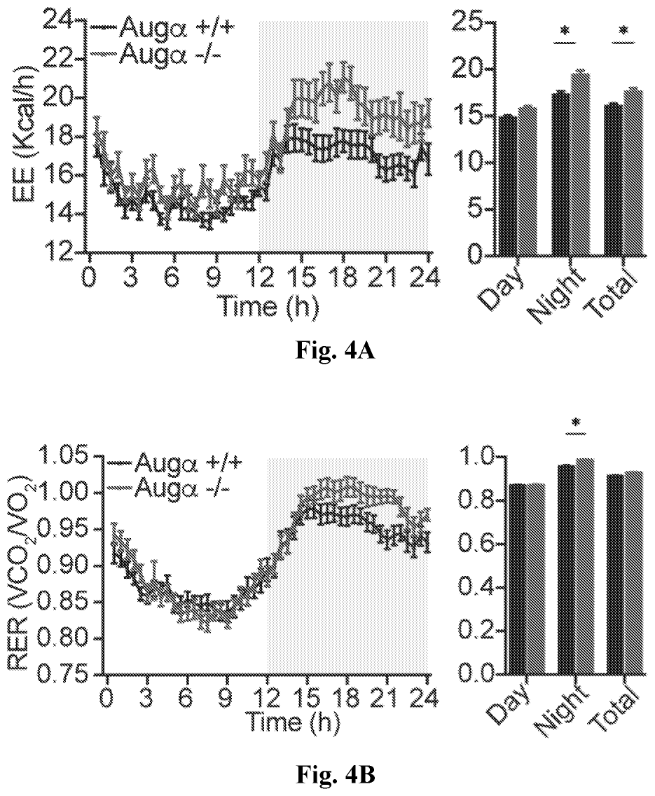

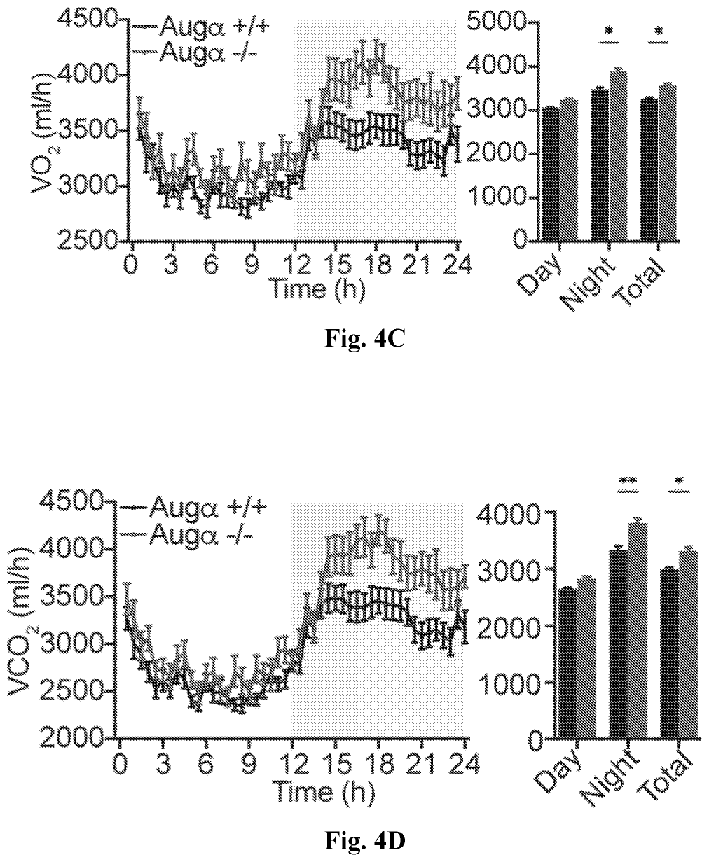

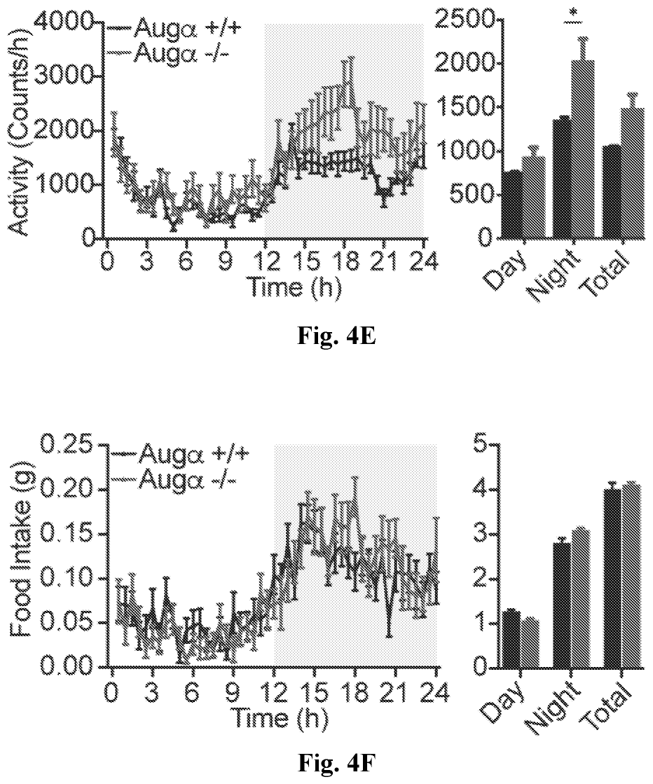

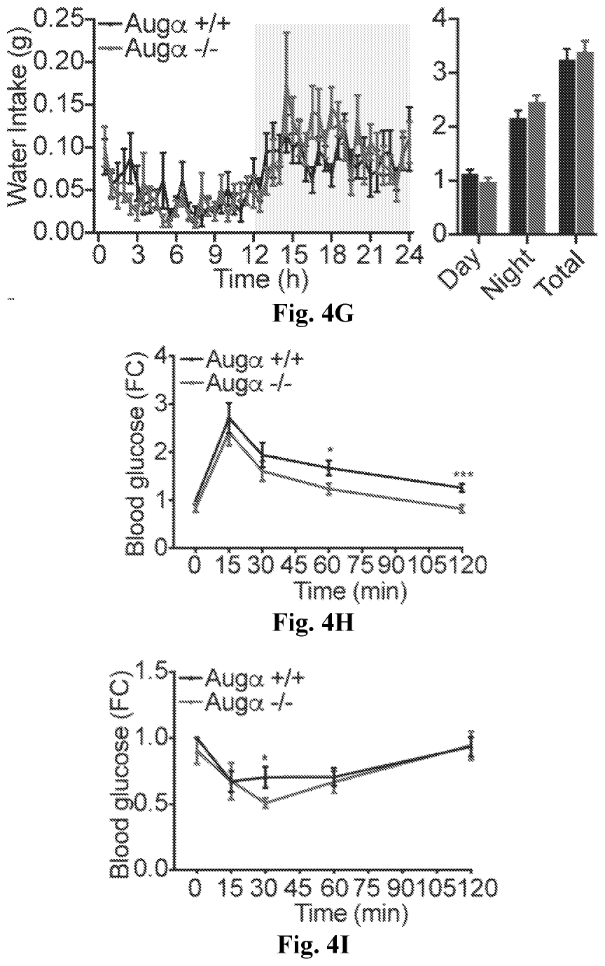

To identify the metabolic parameters responsible for increased fat oxidation and thermogenesis in Augα knockout mice, the present study assessed 3-6 months old Augα knockout and littermate control mice fed on a standard diet in metabolic cages for two days. The present study observed that Augα knockout mice exhibited significantly high energy expenditure, activity, and RER contributed by increased O₂ consumption and CO₂ production (FIGS. 4A-4E). However, no significant changes were detected in food and water intake (FIGS. 4F-4G). Two months old Augα knockout mice had minimal or no change in metabolic parameters (not shown). The present study also assessed these animals for glucose tolerance and insulin sensitivity. The experiment presented in FIGS. 4H-4I show that Augα deficiency leads to improved glucose tolerance and sensitivity to insulin; consistent with increased energy expenditure and activity. The metabolic parameters suggest that the age-dependent thinness of Augα knockout mice is caused by higher energy expenditure and superior glucose clearance due to increased activity.

The metabolic parameters suggest that the age-dependent thinness of Augα knockout mice is caused by higher energy expenditure and superior glucose clearance due to increased activity.

### Example 7: Augα Regulates AgRP Neuronal Projection and Alk Activation in PVN

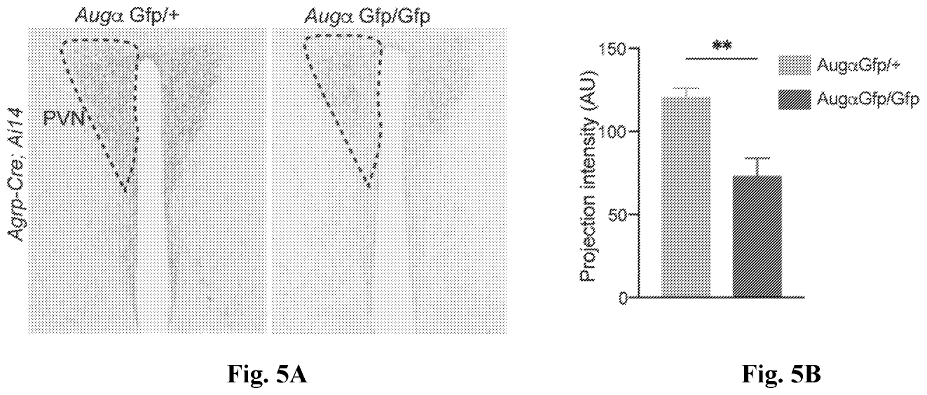

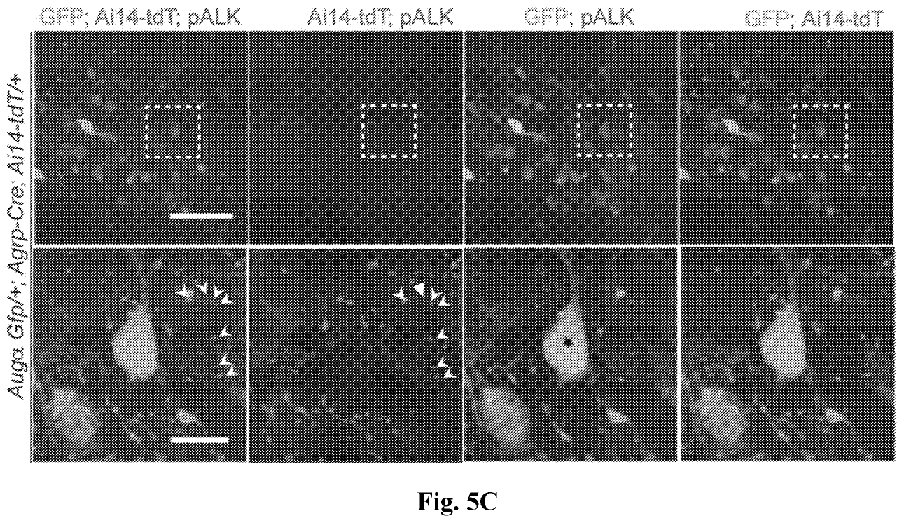

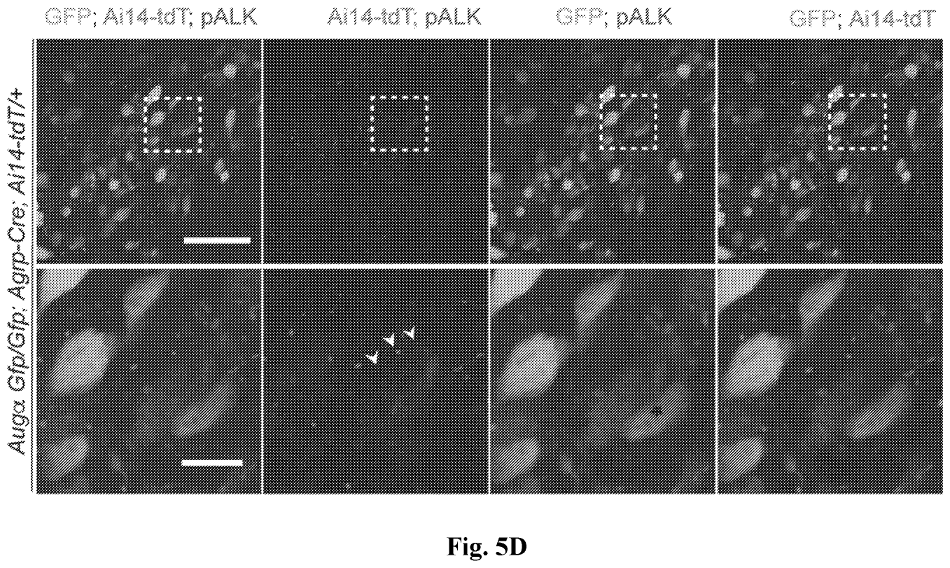

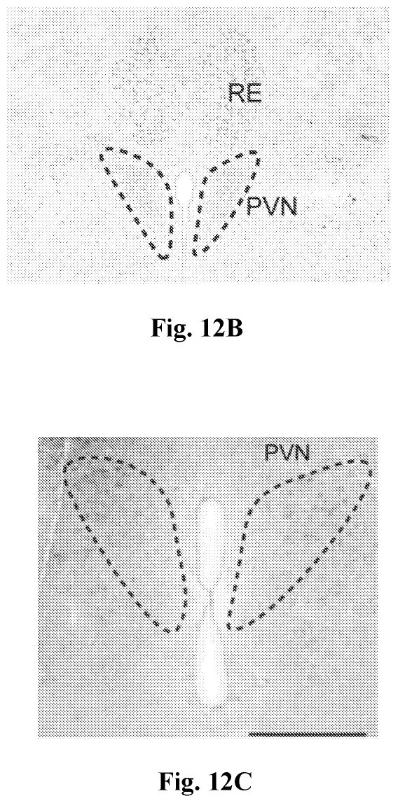

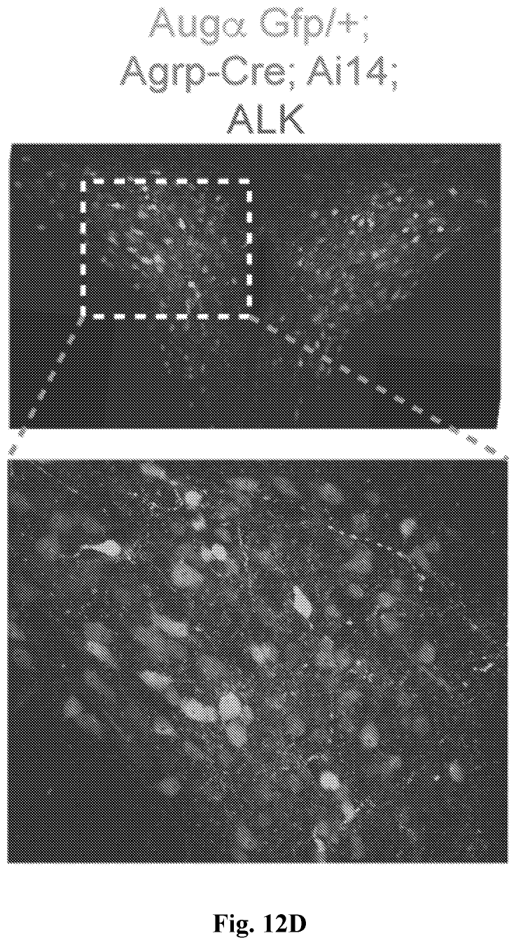

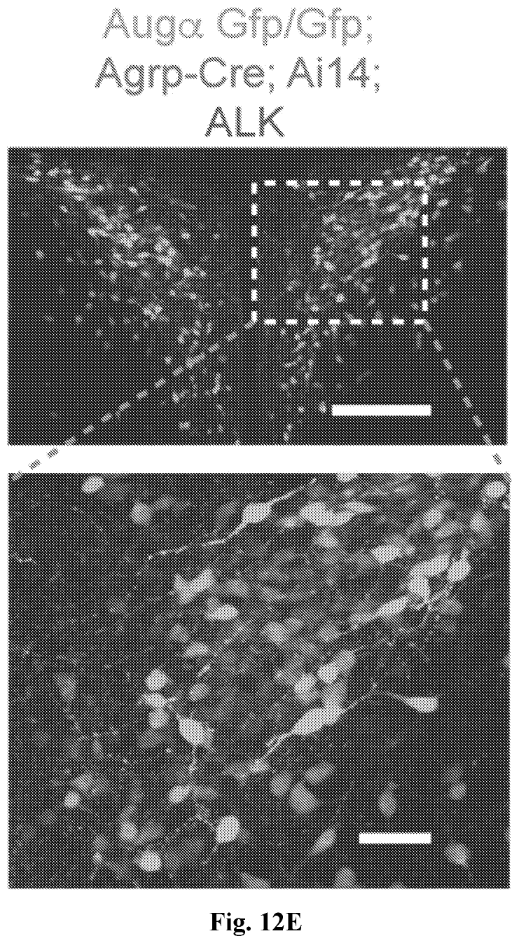

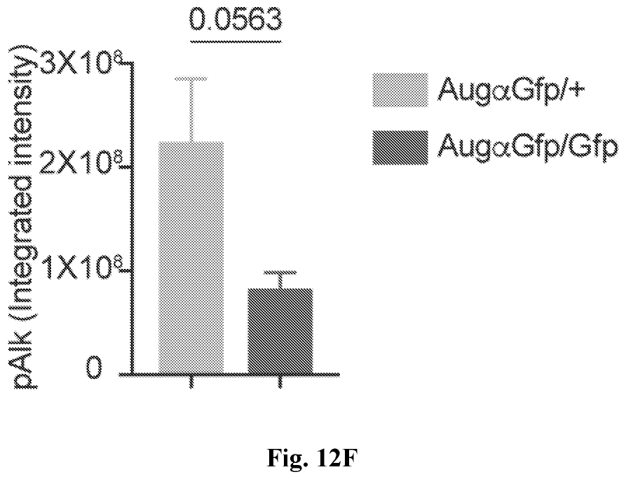

To identify the mechanism of neuronal control of Augα knockout mice thinness, the present study took a cue from the studies where it was showed that Augα induces neurite elongation in neuroblastoma cell lines. To determine whether Augα can also mediate a similar response in mouse neuronal cultures, the present study isolated neural stem cells from a wild-type animal and stimulated them in the presence of recombinant mouse Augα protein. This experiment showed robust increase in neurite formation, as shown in FIG. 12A by staining with MAP2 (Microtubule-associated protein-2). The present study next examined the possibility of whether Augα deletion may affect the arborization of AgRP neuronal projections to PVN, a critical region that regulates energy homeostasis downstream of ARC. In this experiment serial sections of PVN isolated from adult and P15 brains of Augα-Gfp/+; AgRP-Cre; Ai14-tdT and Auga-Gfp/Gfp; AgRP-Cre; Ai14-tdT mice were analyzed for AgRP axon arborization into the PVN. The present study observed that axonal projections of AgRP neurons onto PVN neurons were significantly reduced in numbers indicating a disruption in metabolic circuitry (FIGS. 5A-5D). These results suggest a potential role of Augα in the development of metabolic circuit.

Alk is expressed in a subset of PVN neurons, and that knockout of Alk leads to thinness. Additionally, in situ hybridization data of Alk confirmed Alk localization in PVN (FIGS. 12B-12C). Mechanistically, Augα binding stimulates tyrosine autophosphorylation and Alk activation leading to stimulation of Alk dependent intracellular signaling pathways. To determine if Augα expression in ARC and/or the PVN influences Alk activation within the PVN, the present study performed immunolabelling of Alk using antibodies that bind to Alk extracellular domain, as a measure for Alk level and phospho-Alk (pAlk) selective antibodies as a measure of Alk activation in brain of Augα-Gfp/+; AgRP-Cre; Ai14-tdT and Augα-Gfp/Gfp; AgRP-Cre; Ai14-tdT mice. This experiment showed that tdTomato labelled AgRP projections are in close proximity to pAlk expressing cell bodies suggesting that Augα expressed in AgRP neurons may mediate activation of Alk within the PVN. Moreover, GFP-labelled Augα neurons are in vicinity of the pAlk expressing neurons in PVN, revealing close proximity of endogenous Augα with Alk, consistent with Augα induced Alk activation in these neurons. Further analysis of the Augα-Gfp/Gfp; AgRP-Cre; Ai14-tdT brains revealed that the level of pAlk is reduced in Augα deficient mice (FIGS. 5C-5D and 12F) while the level of total Alk remains unaffected in PVN of Augα deficient mice (FIGS. 12D-12E). These results provide a mechanistic link to earlier finding demonstrating that deletion of Alk from PVN results in thinning of mice. The present study shows that Augα expressed in AgRP and/or the PVN stimulates Alk activation in the PVN, an area within hypothalamus implicated in control of energy metabolism.

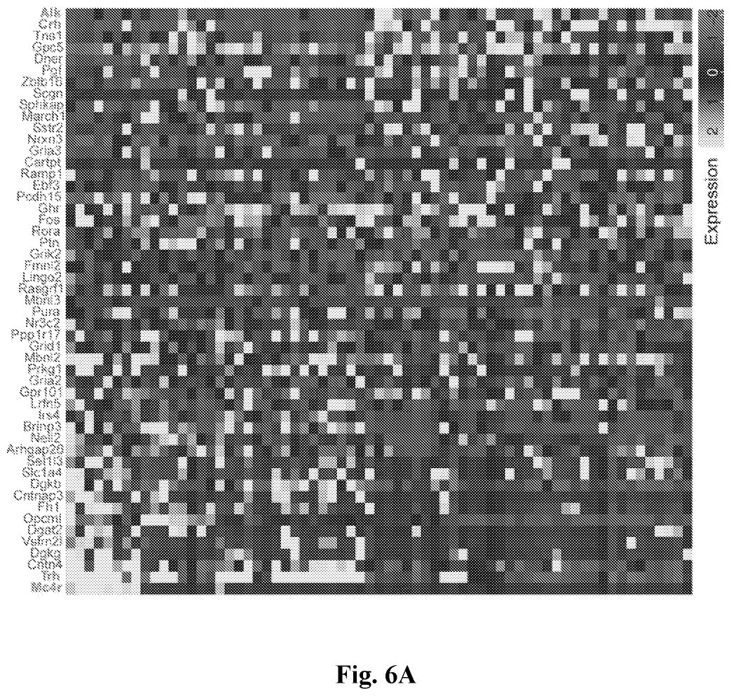

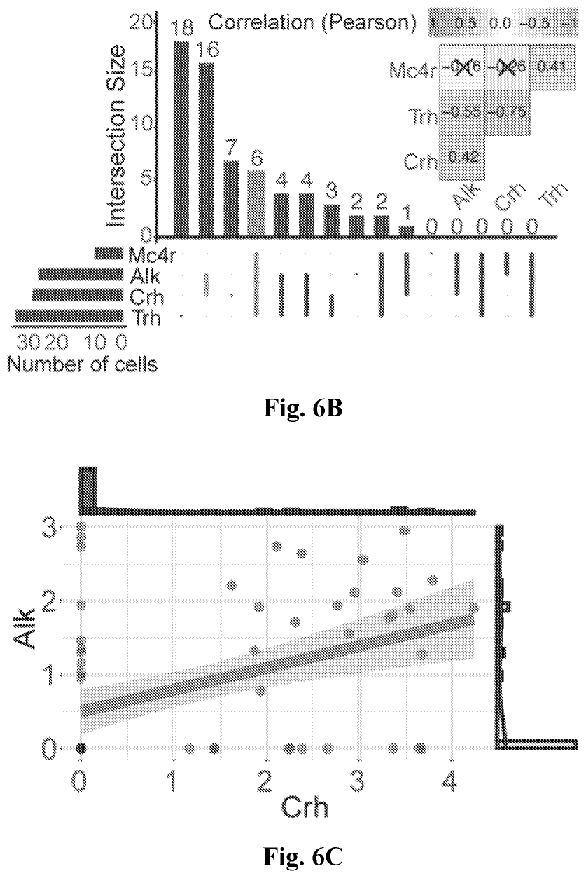

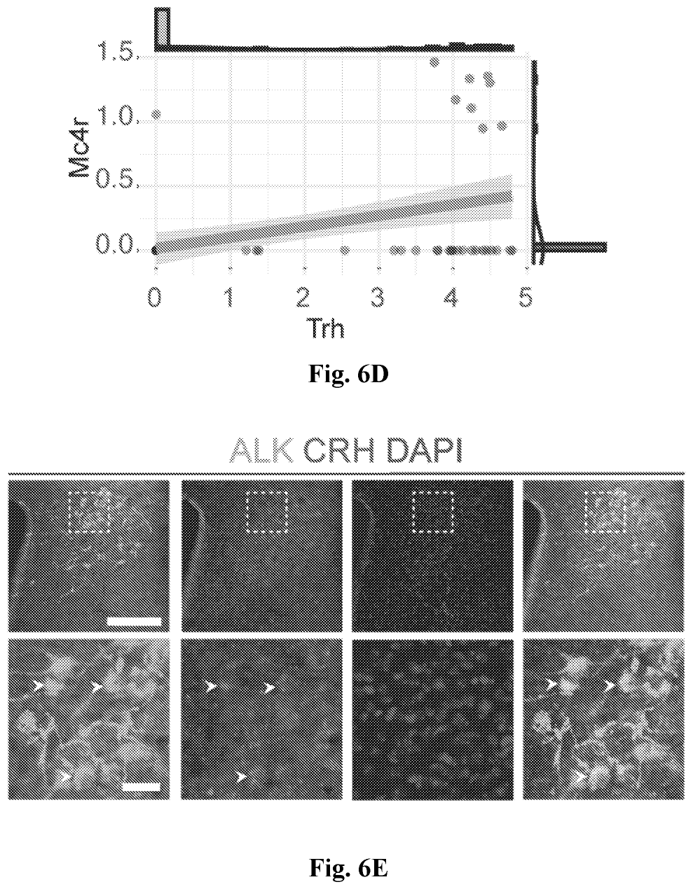

Experiments presented in this manuscript show that Augα activated Alk signaling pathway may operate within the hypothalamus, ARC and PVN neurons, a region in which signaling via MC4 receptors (MC4R) control an important metabolic process that regulate food intake. It was shown that binding of AgRP or melanocyte stimulating hormone (α-MSH) to MC4R in PVN promotes hunger or satiety, respectively and that aberrant activation of this pathway results in severe cases of obesity. To determine whether Augα-Alk signaling pathway is linked to or part of the α-MSH-MC4R signaling pathway, the present study analyzed single-cell RNA-seq data from PVN to identify neurons that express Alk. Interestingly, the data presented in FIGS. 6A-6D show that Alk is not expressed in MC4R expressing neurons which otherwise coexpress thyrotropin release hormone (TRH). Alk, on the other hand, is expressed in corticosterone release hormone (CRH) positive neurons. This result was confirmed by immunostaining analysis (FIG. 6E). These experiments show that MC4R and Alk are expressed in discrete populations of cells within PVN demonstrating that two distinct mechanisms that are also responsible for pigmentation operate within PVN to control body weight.

### Example 8

While it is known that Augα and Augβ bind specifically to the extracellular domains, stimulate tyrosine autophosphorylation of Alk and Ltk and activate multiple intracellular signaling pathways, the physiological roles of Augα and Augβ in mammals are essentially unknown. The present study describes the role played by Augα in the control of brain functions. Experiments are presented demonstrating that Augα is expressed predominantly in AgRP neurons within ARC in hypothalamus and that starvation causes strong Augα expression in AgRP neurons, similar to other orexigenic molecules. It is also demonstrated that Augα deficient mice are thin when either fed a normal or a high fat diet. Upon 16 h of food deprivation, Augα deficient mice displayed increased EE and RER, indicating failed energy conserving mechanisms to cope starvation. Furthermore, upon refeeding after fasting, Augα knockout mice showed a slight decrease in food and water intake. Interestingly, Augα deficient mice fed on standard chow show a significant increase in energy expenditure, respiratory exchange rates and activity without any significant change in food intake, indicating reduced efficiency of the orexigenic pathway to conserve energy. Consistently, Alk deficient mice also show a similar phenotype of thinness attributed to high energy expenditure. Augα deficient mice showed reduced weight gain starting from the age of 3-4 weeks in comparison to their littermate control, and weight differences continued to grow as the mice aged until one year, the last time point recorded. Moreover, the present study showed that Augα deficient mice are thin due to reduced fat content associated with enhanced thermogenesis of white adipose tissue (WAT). Here, the present study demonstrates that Augα expressing AgRP neurons project onto the Alk expressing neurons in PVN and that deletion of Augα leads to suppression of AgRP neurons projection into PVN. Augα expressing AgRP projections in PVN are likely responsible for Alk activation as evident from reduced Alk activation in Augα deficient mice. This result also provides a mechanistic link between Augα stimulation and thinness attributed to the deficiency of Alk expression in PVN. Considering that AgRP neurons are part of the melanocortin system, i.e., AgRP being reverse agonist of MC4 receptors (MC4R), and both Alk and MC4R are expressed in the paraventricular nucleus, the present study showed that the Augα-Alk signaling pathway is innate to but not overlapping with the central melanocortin system. Moreover, it also shows that two distinct mechanisms (a-MSH-MC4R: regulate food intake and Augα-Alk: regulate activity/energy expenditure) playing a central role in control of pigmentation act in concert in the hypothalamus to control body weight. Finally, the present study concludes that the Augα-Alk signaling pathway represents a neuronal signaling pathway controlling metabolic processes that was co-opted to induce a variety of human cancers by aberrant expression of activated Alk mutants.

### Example 9: Material and Methods

### Transgenic Mice Lines

Mice were housed in Yale Animal Resource Center (YARC) controlled facility. Mice were fed ad libitum on a standard chow diet or 45% high fat diet (Research Diets-D12451) and checked daily by veterinary staff. All animals were housed on a 12/12-hour light/dark cycle. For the study, both male and female littermate mice were used unless stated otherwise. All animal experiments were approved by the Institutional Animal Care and Use Committee (IACUC). Augα whole body knockout mice with a Gfp insert on C57BL/6 background were generated through ingenious targeting laboratories. Augα knockout mice with LacZ insert were generated in-house at Yale Genome Editing Center by using targeted ES cells purchased from the European Conditional Mouse Mutagenesis Program (www dot eucomm dot org). Augα knockout mice were then crossed with AgRP-Cre/POMC-Cre/Ai14-tdT mice to label specific neurons for colocalization studies.

### Metabolic Assays

For thinness kinetics, body weights were recorded every week for a year. Body composition of 4-7 months old mice was assessed using an EchoMRI system. Fat and lean mass values were plotted after normalization to body weight. For metabolic parameter, 3-6 months old mice were singly housed and acclimatized in metabolic chambers (TSE Systems) for 2 days, and then metabolic and locomotive parameters as indicated in results were recorded by the build-in automated instruments for another 2 days. Body weight was recorded at the beginning and body weight adjusted values were plotted as indicated in the metabolic cage dataset.

### Glucose, Insulin and Norepinephrine Measurements

7-10 months old mice were deprived of food overnight for glucose tolerance test or 6 h for insulin tolerance test. Glucose level of blood from food deprived mice was measured, and after that either glucose (2.5 g/kg body weight) or Insulin (0.75 U/kg body weight) was administered through intraperitoneal injection. Blood glucose concentrations were measured at the time points as indicated in the data by drawing blood from the tail vein using TRUEtrack glucometer (Trividia Health). Norepinephrine level in adipose tissues and serum was measured using ELISA kit from Abnova.

### Histopathology

Brown and white adipose tissue were dissected from 4-7 months old mice. Mice were anesthetized using isoflourane and the adipose tissue depots were isolated from subcutaneous, gonadal, intraperitoneal and brown adipose depots. Adipose depots were postfixed in 4% formaldehyde overnight. Fixed samples were embedded in paraffin, sectioned, and stained with hematoxylin and eosin staining (H&E) at Yale Histology Core.

### Brain Serial Sections and Immunofluorescence Microscopy

Mice were euthanized with CO₂ and perfused with 4% paraformaldehyde (PFA). Dissected brain tissue was fixed overnight at 4° C. in 4% PFA and embedded in 1.5% low melting agarose (in PBS). The hypothalamic area was sectioned serially into 4 wells of a 12-well plate into 50 μm coronal sections using a vibratome. Sections were stored in 1×PBS+0.02% sodium azide solution. For staining, sections in one well were washed with 1×PBS and incubated with 5% normal donkey serum, 0.3% TWEEN 20 in PBS at RT for 30 mins, incubated with primary antibodies (1:100 to 1:1000 dilution) overnight at 4° C.: Chicken anti-GFP (Aveslabs, GFP-1010), Rabbit Anti-RFP (Rockland, 600-401-379), Goat anti-AgRP (R&D, AF634), Rabbit Anti-POMC (Phoenix pharmaceuticals, H-029-30), Sheep Anti-Alk (R&D, AF4210), Anti-Rabbit-pAlk (Sigma, SAB4504604), Guinea pig Anti-CRH (Peninsula, T-5007) and secondary antibodies (1:200, Alexa Fluor 488 anti-Rabbit IgG, Alexa Fluor 594 anti-Rabbit IgG, and Alexa Fluor 594 anti-Mouse IgG, 1:400) for 1 h. A Leica confocal system was used for fluorescence detection (LSM 800). For quantifications of Augα-Gfp neurons activation after fasting, all the sections on a slide were imaged for hypothalamic areas at 10X and the number of Gfp+neurons within PVN and ARC were counted using Cell Counter Macro of Fiji. The data is plotted as bar graph. Similar quantifications were performed for c-Fos stainings. For pAlk quantifications, mean integrated intensity within PVN was measured using the Fiji software.

### Neurodifferentiation

The cells were harvested and dissociated from PO mice to establish a primary neuronal stem cells culture. Briefly, PO mice were sacrificed, and forebrains were isolated quickly. Using fine forceps, meninges were peeled off; the hippocampi were dissected precisely under a dissecting microscope. The hippocampi were chopped into fine pieces using a sterile scalpel blade and were collected into a 15 ml tube containing a papain-based solution. Tissue was incubated at 37° C. for 15 min and triturated by a fire-polished glass pipette 5-10 times to dissociate the cells. Cells were centrifuged at 300×g for 5 min at room temperature. After washing with 1×PBS twice, cells were plated onto low attachment dishes to propagate neural stem cells population. After 4 days, the neurospheres were plated onto the laminin and poly-D-lysine coated glass coverslips in neurobasal medium supplemented with B27, glutamine and antibiotic with or without recombinant mouse AUG-α protein. After 7 days, the coverslips were stained for MAP2, and images are acquired using LSM 800 (Leica).

### In Situ Hybridization

RNA probes were generated from mouse hypothalamic tissue cDNA as template (Alk, ENSMUST00000086639.6) and in vitro transcribed as per manufacturer's instructions. Probes were purified by phenol/chloroform extraction, quantified and quality controlled and stored at −80° C. till hybridization. Slide-mounted cryo-sections at 30 μm thickness were processed for in situs. Briefly, brains were fixed overnight at 4° C. in 4% PFA diluted in 1×PBS, equilibrated at 4° C. in 30% sucrose in 1×PBS overnight. Fixed brains were then embedded in OCT, sliced on a cryostat (Leica Biosystem). Slides were stored at −80° C. until processed for in situ hybridization. Sections were first postfixed in 4% PFA in 1×PBS for 15 min at RT, washed with 1×PBS, treated with proteinase K and submerged in hybridization buffer (5×SSC, 50% formamide, 1% SDS, 200 mg/ml of aBSA, 500 mg/ml of yeast tRNA and 50 mg/ml of heparin) supplemented with 1000 ng/ml appropriate digoxigenin-labeled probe at 70° C. overnight. Sections were washed two times 45 min at 70° C. in 2×SSC, 50% formamide, 1% SDS, followed by washing in 100 mM Tris HCl pH 7.5, 150 mM NaCl, 0.1% Tween, blocked with 10% sheep inactivated serum (Sigma-Aldrich) and incubated overnight at 4° C. with an anti-digoxigenin antibody conjugated to alkaline phosphatase (1:5000, Roche). Sections were then rinsed in 100 mM Tris-Cl pH 9.5, 100 mM NaCl, 50 mM MgCl₂, 0.1% Tween before being overlaid with BCIP/NBT substrate (Sigma Aldrich). Revelation was done at RT in the dark until the desired signal is reached. Finally, sections were rinsed in 1×PBS, post-fixed with 4% PFA in 1×PBS, washed in water and mounted with paramount medium. The slides were scanned on Aperio CS2 (Leica Biosystems).

RNA Isolation and qPCR

To quantify the expression of thermogenic genes, white adipose tissues from 7-11 months old littermate mice were snap frozen in liquid nitrogen. Tissues were homogenized in Trizol and RNA was extracted with phenol/chloroform, digested with DNase for 15 min followed by cleaned up using PureLink RNA mini kit (Ambion) as per the manufacturer's protocol. cDNA was synthesized using iScript cDNA synthesis kits (Bio-Rad). qPCR reactions were performed in the CFX96 Real-Time PCR Detection System (BioRad) using SYBR supermix (Bio-RAD). Post-amplification melting curve analysis was performed to check for nonspecific products. For normalization, threshold cycles (Ct-values) were normalized to Actin within each sample to obtain sample-specific DCt values (=Ct gene of interest-Ct housekeeping gene). 2∧-DDCt values were calculated to obtain fold expression levels. Values were presented as fold change over littermate control.

### Adipocyte Size

Adipocyte size analyses was performed using adiposoft plugin of image J software on images from H&E-stained slides of white adipose tissues from eight months old mice fed on standard chow.

### Single-Cell RNA-Seq Data Analysis

Pre-processed mouse hypothalamus development scRNA-seq data were downloaded from Gene Expression Omnibus (GEO; accession number: GSE132730). The present study used subset matrices of expression for cluster number 24, which correspond to parvocellular corticotropin-releasing hormone (Crh) and thyrotropin-releasing hormone (Trh) neurons of the paraventricular nucleus of the hypothalamus on postnatal days 10 and 23. Data were processed using the R program environment. Briefly, Seurat R package (v4.0.4; (33)) was used for analysis and visualisation of cellular markers. The present study plotted a heatmap of Pearson's residual values of the genes selected from marker representative for Trh-positive and Crh-positive populations. Differential expression was assessed using the Wilcoxon test, and sorted by correlation with the Trh and Crh genes. Matrices of log-normalised expression values with pseudocount one were used to perform intersection-set analysis with the UpSetR R package (v1.4; (34)), and to examine Pearson correlation statistics, which were visualised using the ggstatsplot R package (v0.8; doi 10.21105/joss.03167).

### Statistical Analysis

All mouse data are expressed as mean±standard error of the mean (SEM). Statistical significance was tested by two tailed upaired student's t test, and one- or two-way ANOVA with Bonferoni corrections for multiple comparison test as indicated in figure legend. All figures and mouse statistical analyses were generated using Prism 8 (GraphPad). In all figures, statistical significance is represented as *p<0.05, ** p<0.01, *** p<0.001, **** p<0.0001.

### Example 10: Pharmaceutical Inhibition of ALK in the Brain Increased Energy Expenditure and Caused Thinness

The present study demonstrated that Augmentor-alpha knock-out mice exhibit thinness phenotype and are resistant to high-fat diet (HFD) induced weight and that Augmentor-alpha and Augmentor-beta function as the physiological ligands of the receptor tyrosine kinases ALK and LTK.

Mutated and activated forms of ALK function as critical mediators of several human cancers and certain cancers metastasize into the brain. Three generations of ALK inhibitors have been developed for the treatment ALK-driven cancers including ALK inhibitors that enter the brain for treatment of ALK driven brain tumors. Crizotinib is a first-generation ALK inhibitor that does not enter the brain. Lorlatinib, a third generation ALK inhibitor, penetrates well into brain.

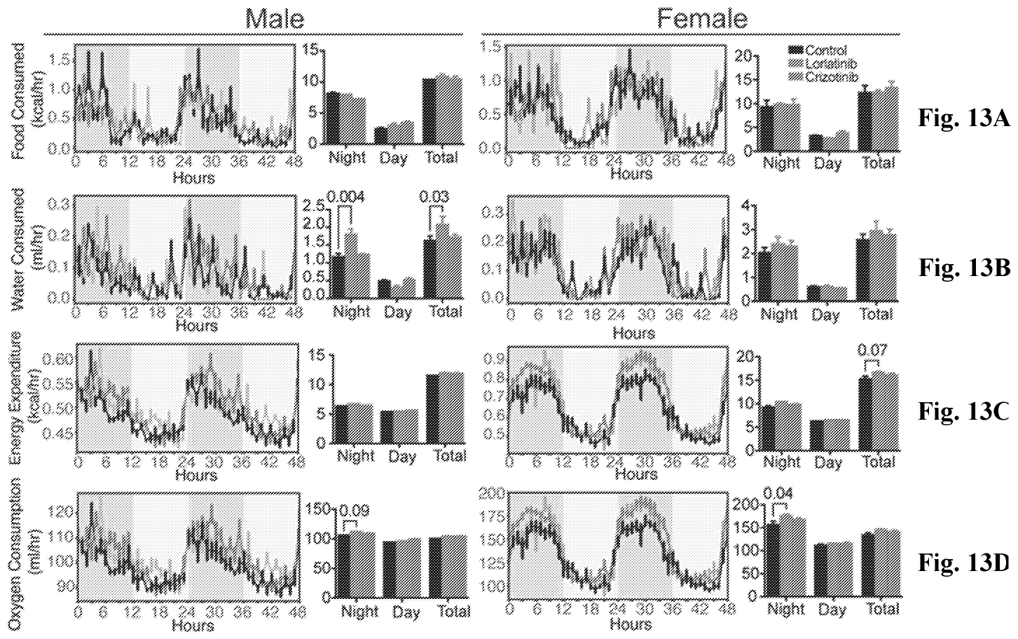

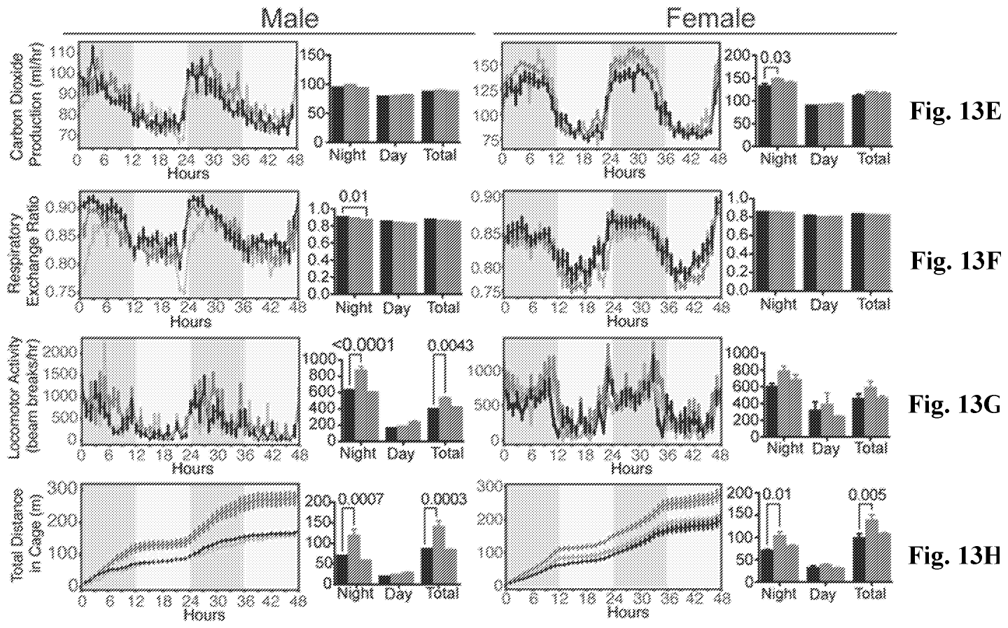

The present study compared the effect of Crizotinib or Lorlatinib treatment on the metabolic parameters of treated mice using sable metabolic cage system. In these experiments three months old male and female mice were singly housed and acclimatized in metabolic chambers for 2 days on HFD. The mice were then injected with either Lorlatininb or Crizotinib at a concentration of 10 mg/kg/day intraperitoneally. For the next two days, data was recorded on food and water intake; Oxygen consumption and CO₂ production; Energy expenditure and respiratory exchange rate; Total distance traveled, and locomotor activity (beam break/hr) (FIGS. 13A-13H). These results were analyzed using CalR, a web-based analysis software for indirect calorimetry experiments (10.1016/j.cmet.2018.06.019). The metabolic cage analyzes showed a significant increase in activity, water intake, CO₂ production, and oxygen consumption, while energy expenditure trended higher without significant changes in food intake in response to Lorlatinib (ALK inhibitor acting in brain) treatment of these mice. By contrast, Crizotinib or vehicle control treatments did not affect metabolic activity of treated mice. These results raise the possibility of repurposing ALK inhibitor cancer drugs for treatment of obesity and other metabolic disorders.

### Enumerated Embodiments

In some aspects, the present invention is directed to the following non-limiting embodiments:

Embodiment 1: A method of increasing energy expenditure levels in a subject in need thereof, wherein the method comprises administering to the subject an effective amount of at least one agent. In certain embodiments, the agent is a small molecule inhibitor of Augmentor α (Augα). In certain embodiments, the agent is a protein inhibitor of Augα. In certain embodiments, the agent is a nucleic acid that downregulates Augα expression level and/or activity by RNA interference, and/or an expression vector expressing the nucleic acid that downregulates Augα expression level and/or activity by RNA interference. In certain embodiments, the agent is a ribozyme that downregulates Augα expression level and/or activity, and/or an expression vector expressing the ribozyme. In certain embodiments, the agent is an expression vector comprising an expression cassette, wherein the expression cassette expresses CRISPR components that downregulate Augα expression level and/or activity by CRISPR knockout or CRISPR knockdown. In certain embodiments, the agent is a trans-dominant negative mutant protein of Augαand/or an expression vector that expresses a trans-dominant negative mutant protein of Augα. In certain embodiments, the agent is a small molecule inhibitor of anaplastic lymphoma kinase (ALK). In certain embodiments, the agent is a protein inhibitor of ALK. In certain embodiments, the agent is a nucleic acid that downregulates ALK expression level and/or activity by RNA interference, and/or an expression vector expressing the nucleic acid that downregulates ALK expression level and/or activity by RNA interference. In certain embodiments, the agent is a ribozyme that downregulates ALK expression level and/or activity, and/or an expression vector expressing the ribozyme. In certain embodiments, the agent is an expression vector comprising an expression cassette, wherein the expression cassette expresses CRISPR components that downregulate ALK expression level and/or activity by CRISPR knockout or CRISPR knockdown. In certain embodiments, the agent is a trans-dominant negative mutant protein of ALK, and/or an expression vector that expresses a trans-dominant negative mutant protein of ALK.

Embodiment 2: The method of Embodiment 1, wherein the level and/or activity of Augα and/or ALK is downregulated in the brain of the subject.

Embodiment 3: The method of any one of Embodiments 1-2, wherein the level and/or activity of Augα and/or ALK is downregulated in the hypothalamus of the brain of the subject.

Embodiment 4: The method of anyone of Embodiments 1-3, wherein the method comprises administering to the subject a small molecule inhibitor of ALK.

Embodiment 5: The method of Embodiment 4, wherein the small molecule inhibitor of ALK is blood brain barrier-penetrating.

Embodiment 6: The method of Embodiment 5, wherein the small molecule inhibitor of ALK comprises Lorlatinib.

Embodiment 7: The method of any one of Embodiment 1-6, wherein the subject is a mammal, optionally a human.

Embodiment 8: A method of reducing body weight in a subject in need thereof, wherein the method comprises administering to the subject an effective amount of at least one agent. In certain embodiments, the agent is a small molecule inhibitor of Augmentor α (Augα). In certain embodiments, the agent is a protein inhibitor of Augα. In certain embodiments, the agent is a nucleic acid that downregulates Augα expression level and/or activity by RNA interference, and/or an expression vector expressing the nucleic acid that downregulates Augα expression level and/or activity by RNA interference. In certain embodiments, the agent is a ribozyme that downregulates Augα expression level and/or activity, and/or an expression vector expressing the ribozyme. In certain embodiments, the agent is an expression vector comprising an expression cassette, wherein the expression cassette expresses CRISPR components that downregulate Augα expression level and/or activity by CRISPR knockout or CRISPR knockdown. In certain embodiments, the agent is a trans-dominant negative mutant protein of Augα, and/or an expression vector that expresses a trans-dominant negative mutant protein of Augα. In certain embodiments, the agent is a small molecule inhibitor of anaplastic lymphoma kinase (ALK). In certain embodiments, the agent is a protein inhibitor of ALK. In certain embodiments, the agent is a nucleic acid that downregulates ALK expression level and/or activity by RNA interference, and/or an expression vector expressing the nucleic acid that downregulates ALK expression level and/or activity by RNA interference. In certain embodiments, the agent is a ribozyme that downregulates ALK expression level and/or activity, and/or an expression vector expressing the ribozyme. In certain embodiments, the agent is an expression vector comprising an expression cassette, wherein the expression cassette expresses CRISPR components that downregulate ALK expression level and/or activity by CRISPR knockout or CRISPR knockdown. In certain embodiments, the agent is a trans-dominant negative mutant protein of ALK, and/or an expression vector that expresses a trans-dominant negative mutant protein of ALK.

Embodiment 9: The method of Embodiment 8, wherein the level and/or activity of Augα and/or ALK is downregulated in the brain of the subject.

Embodiment 10: The method of any one of Embodiments 8-9, wherein the level and/or activity of Augα and/or ALK is downregulated in the hypothalamus of the brain of the subject.

Embodiment 11: The method of any one of Embodiments 8-10, wherein the method comprises administering to the subject a small molecule inhibitor of ALK.

Embodiment 12: The method of Embodiment 11, wherein the small molecule inhibitor of ALK is blood brain barrier-penetrating.

Embodiment 13: The method of Embodiment 12, wherein the small molecule inhibitor of ALK comprises Lorlatinib.

Embodiment 14: The method of any one of Embodiments 8-13, wherein the subject is a mammal, optionally a human.

Embodiment 15: A method of decreasing the amount of white adipose tissue (WAT) and/or promoting browning of WAT in a subject in need thereof, wherein the method comprises administering to the subject an effective amount of at least one agent. In certain embodiments, the agent is a small molecule inhibitor of Augmentor α (Augα). In certain embodiments, the agent is a protein inhibitor of Augα. In certain embodiments, the agent is a nucleic acid that downregulates Augα expression level and/or activity by RNA interference, and/or an expression vector expressing the nucleic acid that downregulates Augα expression level and/or activity by RNA interference. In certain embodiments, the agent is a ribozyme that downregulates Augα expression level and/or activity, and/or an expression vector expressing the ribozyme. In certain embodiments, the agent is an expression vector comprising an expression cassette, wherein the expression cassette expresses CRISPR components that downregulate Augα expression level and/or activity by CRISPR knockout or CRISPR knockdown. In certain embodiments, the agent is a trans-dominant negative mutant protein of Augα, and/or an expression vector that expresses a trans-dominant negative mutant protein of Augα. In certain embodiments, the agent is a small molecule inhibitor of anaplastic lymphoma kinase (ALK). In certain embodiments, the agent is a protein inhibitor of ALK. In certain embodiments, the agent is a nucleic acid that downregulates ALK expression level and/or activity by RNA interference, and/or an expression vector expressing the nucleic acid that downregulates ALK expression level and/or activity by RNA interference. In certain embodiments, the agent is a ribozyme that downregulates ALK expression level and/or activity, and/or an expression vector expressing the ribozyme. In certain embodiments, the agent is an expression vector comprising an expression cassette, wherein the expression cassette expresses CRISPR components that downregulate ALK expression level and/or activity by CRISPR knockout or CRISPR knockdown. In certain embodiments, the agent is a trans-dominant negative mutant protein of ALK, and/or an expression vector that expresses a trans-dominant negative mutant protein of ALK.a nucleic acid that downregulates the expression level and/or activity of Augα by RNA interference, and/or an expression vector expressing the nucleic acid that downregulates the expression level and/or activity of Augα by RNA interference.

Embodiment 16: The method of Embodiment 15, wherein the level and/or activity of Augα and/or ALK is downregulated in the brain of the subject.

Embodiment 17: The method of any one of Embodiments 15-16, wherein the level and/or activity of Augα and/or ALK is downregulated in the hypothalamus of the brain of the subject.

Embodiment 18: The method of any one of Embodiments 15-17, wherein the method comprises administering to the subject a small molecule inhibitor of ALK.

Embodiment 19: The method of Embodiment 18, wherein the small molecule inhibitor of ALK is blood brain barrier-penetrating.

Embodiment 20: The method of Embodiment 19, wherein the small molecule inhibitor of ALK comprises Lorlatinib.

Embodiment 21: The method of any one of Embodiments 15-20, wherein the subject is a mammal, optionally a human.

Embodiment 22: A method of improving glucose tolerance and/or insulin sensitivity in a subject in need thereof, the method comprising administering to the subject an effective amount of at least one agent. In certain embodiments, the agent is a small molecule inhibitor of Augmentor α (Augα). In certain embodiments, the agent is a protein inhibitor of Augα. In certain embodiments, the agent is a nucleic acid that downregulates Augα expression level and/or activity by RNA interference, and/or an expression vector expressing the nucleic acid that downregulates Augα expression level and/or activity by RNA interference. In certain embodiments, the agent is a ribozyme that downregulates Augα expression level and/or activity, and/or an expression vector expressing the ribozyme. In certain embodiments, the agent is an expression vector comprising an expression cassette, wherein the expression cassette expresses CRISPR components that downregulate Augα expression level and/or activity by CRISPR knockout or CRISPR knockdown. In certain embodiments, the agent is a trans-dominant negative mutant protein of Augα, and/or an expression vector that expresses a trans-dominant negative mutant protein of Augα. In certain embodiments, the agent is a small molecule inhibitor of anaplastic lymphoma kinase (ALK). In certain embodiments, the agent is a protein inhibitor of ALK. In certain embodiments, the agent is a nucleic acid that downregulates ALK expression level and/or activity by RNA interference, and/or an expression vector expressing the nucleic acid that downregulates ALK expression level and/or activity by RNA interference. In certain embodiments, the agent is a ribozyme that downregulates ALK expression level and/or activity, and/or an expression vector expressing the ribozyme. In certain embodiments, the agent is an expression vector comprising an expression cassette, wherein the expression cassette expresses CRISPR components that downregulate ALK expression level and/or activity by CRISPR knockout or CRISPR knockdown. In certain embodiments, the agent is a trans-dominant negative mutant protein of ALK, and/or an expression vector that expresses a trans-dominant negative mutant protein of ALK.a small molecule inhibitor of Augα.

Embodiment 23: The method of Embodiment 22, wherein the level and/or activity of Augα and/or ALK is downregulated in the brain of the subject.

Embodiment 24: The method of any one of Embodiments 22-23, wherein the level and/or activity of Augα and/or ALK is downregulated in the hypothalamus of the brain of the subject.

Embodiment 25: The method of any one of Embodiments 22-24, wherein the method comprises administering to the subject a small molecule inhibitor of ALK.

Embodiment 26: The method of Embodiment 25, wherein the small molecule inhibitor of ALK is blood brain barrier-penetrating.

Embodiment 27: The method of Embodiment 26, wherein the small molecule inhibitor of ALK comprises Lorlatinib.

Embodiment 28: The method of Embodiments 21-27, wherein the subject is a mammal, optionally a human.

Embodiment 29: A method of decreasing energy expenditure levels in a subject in need thereof, the method comprising administering to the subject an effective amount of at least one agent. In certain embodiments, the agent is Augα, or a fragment thereof. In certain embodiments, the agent is a modified Augα, or a fragment thereof. In certain embodiments, the agent is an expression vector expressing the Augα, modified Augα, or fragment thereof. In certain embodiments, the agent is ALK, or a fragment thereof. In certain embodiments, the agent is a modified ALK, or a fragment thereof. In certain embodiments, the agent is an expression vector expressing the ALK, modified ALK, or fragment thereof.

Embodiment 30: The method of Embodiment 29, wherein the level and/or activity of Augα and/or ALK is upregulated in the brain of the subject.

Embodiment 31: The method of any one of Embodiments 29-30, wherein the level and/or activity of Augα and/or ALK is upregulated in the hypothalamus of the brain of the subject.

Embodiment 32: The method of any one of Embodiments 29-31, wherein the subject is administered with a biologically active fragment of Augα.

Embodiment 33: The method of any one of Embodiments 29-32, wherein the subject is a mammal, optionally a human.

Embodiment 34: A method of increasing body weight in a subject in need thereof, the method comprising administering to the subject an effective amount of at least one agent. In certain embodiments, the agent is Augα, or a fragment thereof. In certain embodiments, the agent is a modified Augα, or a fragment thereof. In certain embodiments, the agent is an expression vector expressing the Augα, modified Augα, or fragment thereof. In certain embodiments, the agent is ALK, or a fragment thereof. In certain embodiments, the agent is a modified ALK, or a fragment thereof. In certain embodiments, the agent is an expression vector expressing the ALK, modified ALK, or fragment thereof.

Embodiment 35: The method of Embodiment 34, wherein the level and/or activity of Augα and/or ALK is upregulated in the brain of the subject.

Embodiment 36: The method of any one of Embodiments 34-35, wherein the level and/or activity of Augα and/or ALK is upregulated in the hypothalamus of the brain of the subject.

Embodiment 37: The method of any one of Embodiments 34-36, wherein the subject is administered a biologically active fragment of Augα.

Embodiment 38: The method of any one of Embodiments 34-38, wherein the subject is a mammal, optionally a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of increasing energy expenditure levels in a subject in need thereof,
wherein the method comprises administering to the subject an effective amount of at least one selected from the group consisting of:
a small molecule inhibitor of Augmentor α (Augα),
a protein inhibitor of Augα,
a nucleic acid that downregulates Augα level or activity by RNA interference, or an expression vector expressing the nucleic acid that downregulates Augα level or activity by RNA interference,
a ribozyme that downregulates Augα level or activity, or an expression vector expressing the ribozyme,
an expression vector comprising an expression cassette, wherein the expression cassette expresses CRISPR components that downregulate Augα level or activity by CRISPR knockout or CRISPR knockdown, and
a trans-dominant negative mutant protein of Augα, or an expression vector that expresses a trans-dominant negative mutant protein of Augα;
or
wherein the method comprises administering to the subject an effective amount of at least one selected from the group consisting of:
a small molecule inhibitor of anaplastic lymphoma kinase (ALK),
a protein inhibitor of ALK,
a nucleic acid that downregulates ALK level or activity by RNA interference, or an expression vector expressing the nucleic acid that downregulates ALK level or activity by RNA interference,
a ribozyme that downregulates ALK level or activity, or an expression vector expressing the ribozyme,
an expression vector comprising an expression cassette, wherein the expression cassette expresses CRISPR components that downregulate ALK level or activity by CRISPR knockout or CRISPR knockdown, and
a trans-dominant negative mutant protein of ALK, or an expression vector that expresses a trans-dominant negative mutant protein of ALK.

2. The method of claim 1, wherein the level or activity of Augα or ALK is downregulated in the brain of the subject, optionally in the hypothalamus of the brain of the subject.

3. (canceled)

4. The method of claim 1, wherein the method comprises administering to the subject a small molecule inhibitor of ALK, optionally wherein the small molecule inhibitor of ALK is blood brain barrier-penetrating.

5. (canceled)

6. The method of claim 5, wherein the small molecule inhibitor of ALK comprises Lorlatinib.

7. The method of claim 1, wherein the subject is a mammal, optionally a human.

8. A method of reducing body weight in a subject in need thereof,
wherein the method comprises administering to the subject an effective amount of at least one selected from the group consisting of:
a small molecule inhibitor of Augα,
a protein inhibitor of Augα,
a nucleic acid that downregulates the expression level or activity of Augα by RNA interference, or an expression vector expressing the nucleic acid that downregulates the expression level or activity of Augα by RNA interference,
a ribozyme that downregulates the expression level or activity of Augα, or an expression vector expressing the ribozyme,
an expression vector comprising an expression cassette, wherein the expression cassette expresses CRISPR components that downregulate the expression level or activity of Augα by CRISPR knockout or CRISPR knockdown, and
a trans-dominant negative mutant protein of Augα, or an expression vector that expresses the trans-dominant negative mutant protein of Augα;
or
wherein the method comprises administering to the subject an effective amount of at least one selected from the group consisting of:
a small molecule inhibitor of anaplastic lymphoma kinase (ALK),
a protein inhibitor of ALK,
a nucleic acid that downregulates ALK level or activity by RNA interference, or an expression vector expressing the nucleic acid that downregulates ALK level or activity by RNA interference,
a ribozyme that downregulates ALK level or activity, or an expression vector expressing the ribozyme,
an expression vector comprising an expression cassette, wherein the expression cassette expresses CRISPR components that downregulate ALK level or activity by CRISPR knockout or CRISPR knockdown, and
a trans-dominant negative mutant protein of ALK, or an expression vector that expresses a trans-dominant negative mutant protein of ALK.

9. The method of claim 8, wherein the level or activity of Augα or ALK is downregulated in the brain of the subject, optionally in the hypothalamus of the brain of the subject.

10. (canceled)

11. The method of claim 8, wherein the method comprises administering to the subject a small molecule inhibitor of ALK, optionally wherein the small molecule inhibitor of ALK is blood brain barrier-penetrating.

12. (canceled)

13. The method of claim 11, wherein the small molecule inhibitor of ALK comprises Lorlatinib.

14. The method of claim 8, wherein the subject is a mammal, optionally a human.

15. A method of decreasing the amount of white adipose tissue (WAT) or promoting browning of WAT in a subject in need thereof,
wherein the method comprises administering to the subject an effective amount of at least one selected from the group consisting of:
a small molecule inhibitor of Augα,
a protein inhibitor of Augα,
a nucleic acid that downregulates the expression level or activity of Augα by RNA interference, or an expression vector expressing the nucleic acid that downregulates the expression level or activity of Augα by RNA interference,
a ribozyme that downregulates the expression level or activity of Augα, or an expression vector expressing the ribozyme,
an expression vector comprising an expression cassette, wherein the expression cassette expresses CRISPR components that downregulate the expression level and/or activity of Augα by CRISPR knockout or CRISPR knockdown, and
a trans-dominant negative mutant protein of Augα, or an expression vector that expresses the trans-dominant negative mutant protein of Augα;
or
wherein the method comprises administering to the subject an effective amount of at least one selected from the group consisting of:
a small molecule inhibitor of anaplastic lymphoma kinase (ALK),
a protein inhibitor of ALK,
a nucleic acid that downregulates ALK level or activity by RNA interference, or an expression vector expressing the nucleic acid that downregulates ALK level or activity by RNA interference,
a ribozyme that downregulates ALK level or activity, or an expression vector expressing the ribozyme,
an expression vector comprising an expression cassette, wherein the expression cassette expresses CRISPR components that downregulate ALK level or activity by CRISPR knockout or CRISPR knockdown, and
a trans-dominant negative mutant protein of ALK, or an expression vector that expresses a trans-dominant negative mutant protein of ALK.

16. The method of claim 15, wherein the level or activity of Augα or ALK is downregulated in the brain of the subject, optionally in the hypothalamus of the brain of the subject.

17. (canceled)

18. The method of claim 15, wherein the method comprises administering to the subject a small molecule inhibitor of ALK, optionally wherein the small molecule inhibitor of ALK is blood brain barrier-penetrating.

19. (canceled)

20. The method of claim 18, wherein the small molecule inhibitor of ALK comprises Lorlatinib.

21. The method of claim 15, wherein the subject is a mammal, optionally a human.

22. A method of improving glucose tolerance and/or insulin sensitivity in a subject in need thereof,
the method comprising administering to the subject an effective amount of at least one selected from the group consisting of:
a small molecule inhibitor of Augα,
a protein inhibitor of Augα,
a nucleic acid that downregulates the expression level or activity of Augα by RNA interference, or an expression vector expressing the nucleic acid that downregulates the expression level or activity of Augα by RNA interference,
a ribozyme that downregulates the expression level or activity of Augα, or an expression vector expressing the ribozyme,
an expression vector comprising an expression cassette, wherein the expression cassette expresses CRISPR components that downregulate the expression level or activity of Augα by CRISPR knockout or CRISPR knockdown, and
a trans-dominant negative mutant protein of Augα, or an expression vector that expresses the trans-dominant negative mutant protein of Augα;
or
wherein the method comprises administering to the subject an effective amount of at least one selected from the group consisting of:
a small molecule inhibitor of anaplastic lymphoma kinase (ALK),
a protein inhibitor of ALK,
a nucleic acid that downregulates ALK level or activity by RNA interference, or an expression vector expressing the nucleic acid that downregulates ALK level or activity by RNA interference,
a ribozyme that downregulates ALK level or activity, or an expression vector expressing the ribozyme,
an expression vector comprising an expression cassette, wherein the expression cassette expresses CRISPR components that downregulate ALK level or activity by CRISPR knockout or CRISPR knockdown, and
a trans-dominant negative mutant protein of ALK, or an expression vector that expresses a trans-dominant negative mutant protein of ALK.

23. The method of claim 22, wherein the level or activity of Augα or ALK is downregulated in the brain of the subject, optionally in the hypothalamus of the brain of the subject.

24. (canceled)

25. The method of claim 22, wherein the method comprises administering to the subject a small molecule inhibitor of ALK, optionally wherein the small molecule inhibitor of ALK is blood brain barrier-penetrating.

26. (canceled)

27. The method of claim 25, wherein the small molecule inhibitor of ALK comprises Lorlatinib.

28. The method of claim 21, wherein the subject is a mammal, optionally a human.

29. A method of decreasing energy expenditure levels in a subject in need thereof,
the method comprising administering to the subject:
an effective amount of at least one selected from the group consisting of Augα, a
modified Augα, or a fragment thereof, or an expression vector expressing the Augα, modified Augα, or fragment thereof;
or
an effective amount of at least one selected from the group consisting of ALK, a modified ALK, or a fragment thereof, or an expression vector expressing the ALK, modified ALK, or fragment thereof.

30. The method of claim 29, wherein the level or activity of Augα or ALK is upregulated in the brain of the subject, optionally in the hypothalamus of the brain of the subject.

31. (canceled)

32. The method of claim 29, wherein the subject is administered with a biologically active fragment of Augα.

33. The method of claim 29, wherein the subject is a mammal, optionally a human.

34. A method of increasing body weight in a subject in need thereof, the method comprising administering to the subject:
an effective amount of at least one selected from the group consisting of Augα, a modified Augα, or a fragment thereof, or an expression vector expressing the Augα, modified Augα, or fragment thereof,
or
an effective amount of at least one selected from the group consisting of ALK, a modified ALK, or a fragment thereof, or an expression vector expressing the ALK, modified ALK, or fragment thereof.

35. The method of claim 34, wherein the level or activity of Augα or ALK is upregulated in the brain of the subject, optionally in the hypothalamus of the brain of the subject.

36. (canceled)

37. The method of claim 34, wherein the subject is administered a biologically active fragment of Augα.

38. The method of claim 34, wherein the subject is a mammal, optionally a human.
